(19) United States
(12) Patent Application Publication
(10) Pub. No.: US 2014/0323705 A1
(43) Pub. Date: Oct. 30, 2014

(54) MANUFACTURE OF LACTO-N-TETRAOSE

(71) Applicants: Markus Hederos, Svedala (SE); Gyula Dekany, Sinnamon Park (AU); Sándor Demkó, Debrecen (HU); Imre Kovács, Debreceb (HU); István Bajza, Debrecen (HU)

(72) Inventors: Markus Hederos, Svedala (SE); Gyula Dekany, Sinnamon Park (AU); Sándor Demkó, Debrecen (HU); Imre Kovács, Debreceb (HU); István Bajza, Debrecen (HU)

(21) Appl. No.: 14/117,104

(22) PCT Filed: May 14, 2012
(86) PCT No.: PCT/DK12/50170

§ 371 Date: Nov. 12, 2013

(30) Foreign Application Priority Data
May 13, 2011 (EP) 11166001.5

(51) Int. Cl. C07H 15/20; C07H 1/00

(52) U.S. Cl. CPC C07H 15/20; C07H 1/00
USPC 536/17.2

(57) ABSTRACT

The present invention relates to the synthesis of the tetrasaccharide of formula (I) and novel intermediates used in the synthesis.

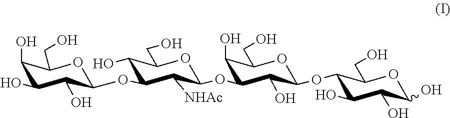

### FIELD OF INVENTION

The present invention relates to a method for the manufacture of Galpβ1-3GlcNAcpβ1-3Galpβ1-4Glc (lacto-N-tetraose, LNT) and starting materials/intermediates for the manufacture of LNT.

### BACKGROUND OF INVENTION

An importance of HMOs is directly linked to their unique biological activities such as antibacterial, antiviral, immune system and cognitive development enhancing activities.

During the past decades an interest for the manufacture and commercialisation of human milk oligosaccharides (HMOs) has been steadily increasing.

A tetrasaccharide Galpβ1-3GlcNAcpβ1-3Galpβ1-4Glc (lacto-N-tetraose, LNT, Scheme 1) is one of the oligosaccharides occurring in human milk [Kuhn et al. Chem. Ber. 1953, 86, 827]. The tetrasaccharide LNT acts as bacterial receptor for pneumococci and was found to be useful in the recognition of the acceptor specificity of glycosyltransferases, the substrate specificity of glycosidases and the structure of antigenic determinants.

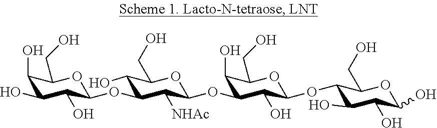

LNT represents a core structure of more complex human milk oligosaccharides, in glycolipids and in glycoproteins having various physiological activities.

To date, it has not been possible to provide routes to access large volumes of LNT. Such problems are not readily overcome by using various isolation, biotechnology and synthetic methodologies. The isolation of LNT from human milk is rather difficult, even in milligram quantities due to the presence of a large number of similar oligosaccharides which means that isolation techniques are difficult.

Methods for the manufacture of LNT are known. For example Takamura et al. Chem. Pharm. Bull. 1979, 27, 1497 and 1980, 28, 1804; Aly et al. Carbohydr. Res. 1999, 316, 121; Malleron et al. ibid. 2008, 343, 970 describe methods which comprise a large number of reaction steps, protecting group manipulations and chromatographic purification. Such methods provide poor yields and provide small amount of LNT, thus these methods do not offer attractive techniques for large scale preparation.

Benzyl glycoside of LNT has been synthesized using chemical or enzymatical means in Malleron et al. Carbohydr. Res. 2006, 341, 29 and Liu et al. Bioorg. Med. Chem. 2009, 17, 4910, respectively.

With respect to LNT and intermediates thereof, there is a need for crystalline products which may simplify isolation, purification and formulation problems. There is also a need to be able to manufacture such compounds and intermediated thereof on a large scale.

### BRIEF DESCRIPTION OF FIGURES

FIG. 1 shows the overview of the manufacture LNT according to the present invention.

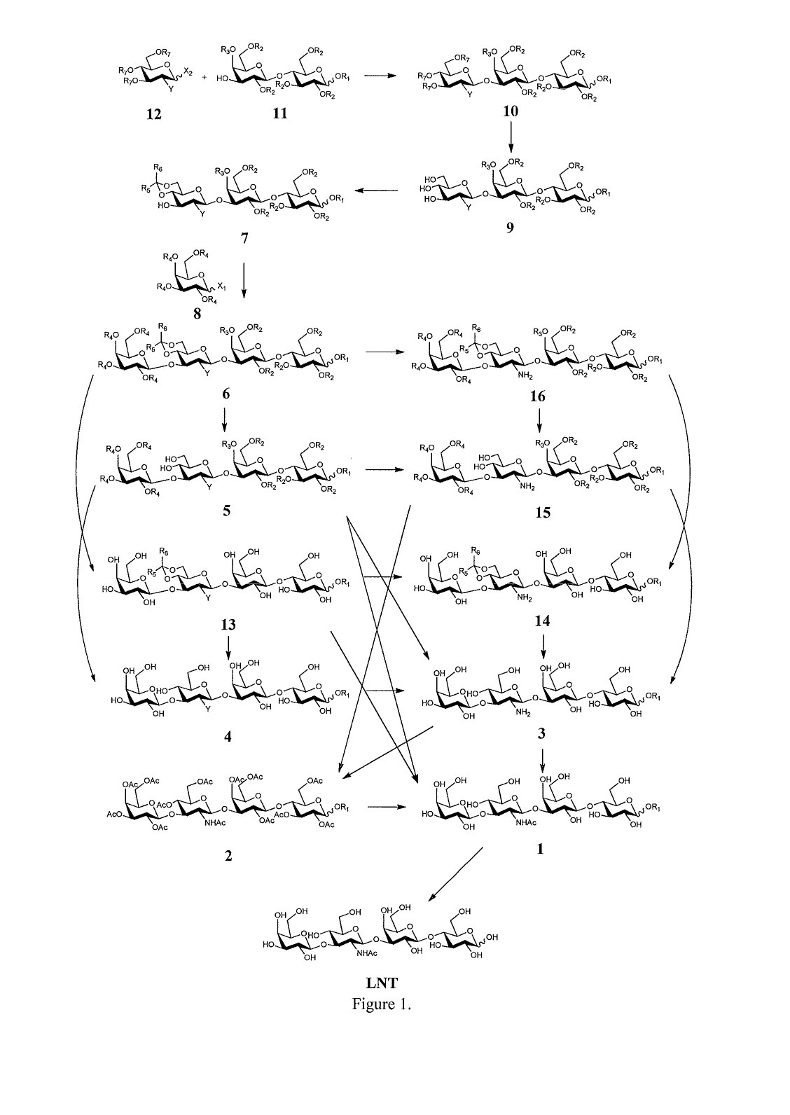

### SUMMARY OF INVENTION

The first aspect of the invention relates to a method for the manufacture of Galpβ1-3GlcNAcpβ1-3Galpβ1-4Glc (LNT), comprising a catalytic hydrogenolysis of a compound of general formula 1

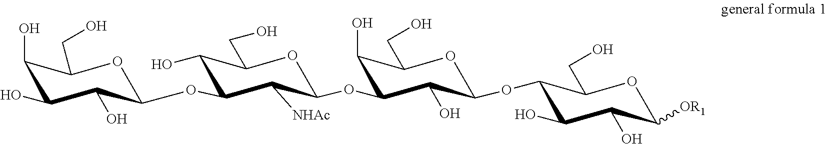

wherein R₁ is a group removable by catalytic hydrogenolysis.

According to an embodiment of the first aspect, the compound of general formula 1 is obtained by a conversion of a compound of general formula 6

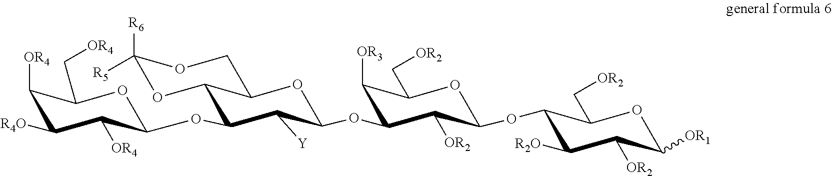

wherein R₁ is a group removable by catalytic hydrogenolysis,
R₂ is optionally substituted acyl,
R₃ is H or optionally substituted acyl,
R₄ is optionally substituted acyl,
R₅ is alkyl or optionally substituted phenyl,
R₆ is H, alkyl or optionally substituted phenyl, or
R₅ and R₆ with the carbon atom to which they are attached form a C₃-C₆ cycloalkyl ring, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
   by deprotection of R₂, R₃, R₄ and moiety

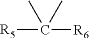

and by transformation of Y.

According to an embodiment of the first aspect, the compound of general formula 1 is obtained from a compound of general formula 6 comprising:
a) an acid catalyzed hydrolysis of the compound of general formula 6 to a compound of general formula 5

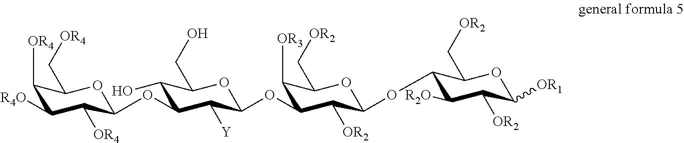

wherein R₁ is a group removable by catalytic hydrogenolysis,
R₂ is optionally substituted acyl,
R₃ is H or optionally substituted acyl,
R₄ is optionally substituted acyl, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido, then
b) a further conversion of the compound of general formula 5 obtained above by deprotection of R₂, R₃ and R₄, and by transformation of Y to form the compound of general formula 1.

According to an embodiment of the first aspect, the compound of general formula 1 is obtained by a based catalyzed transesterification or a basic hydrolysis from a compound of general formula 5

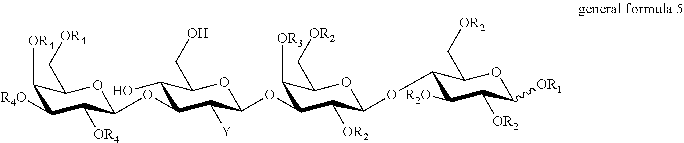

when R₁ is a group removable by catalytic hydrogenolysis,
R₂ is optionally substituted acyl,
R₃ is H or optionally substituted acyl,
R₄ is optionally substituted acyl, and
Y is —NHAc or —NAc₂.

According to an embodiment of the first aspect, the compound of general formula 1 is obtained from a compound of general formula 5 comprising:
a) a conversion of a compound of general formula 5

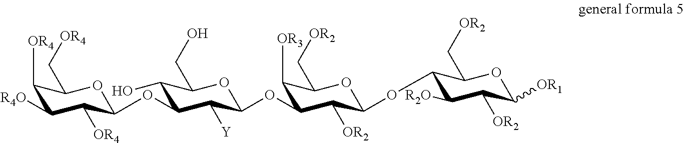

wherein R₁ is a group removable by catalytic hydrogenolysis,
R₂ is optionally substituted acyl,
R₃ is H or optionally substituted acyl,
R₄ is optionally substituted acyl, and
Y is selected from alkanoylamido, haloalkanoylamido, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
by deprotection of R₂, R₃, R₄ and Y to form a compound of general formula 3

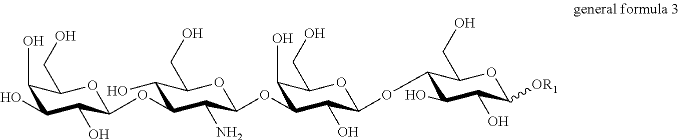

wherein R₁ is a group removable by catalytic hydrogenolysis, and
b) a conversion of the compound of general formula 3 into the compound of general formula 1 by:
   ba) a selective N-acetylation of the compound of general formula 3, or
   bb) a peracetylation of the compound of general formula 3 to a compound of general formula 2

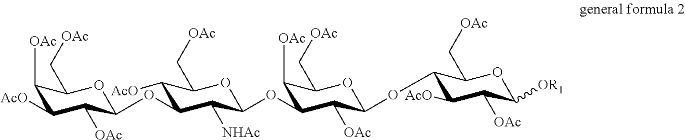

wherein R₁ is as defined above,
   followed by a base catalysed transesterification or a basic hydrolysis.

According to an embodiment of the first aspect, the compound of general formula 6 is made in the reaction of a donor of general formula 8

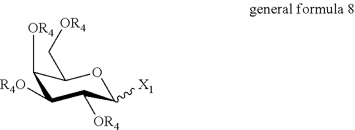

wherein R₄ is optionally substituted acyl, and
X₁ is selected from halogen, —OC(═NH)CCl₃, —OAc, —OBz and —SR₇, wherein R₇ is selected from alkyl and optionally substituted phenyl,
   with an acceptor of general formula 7

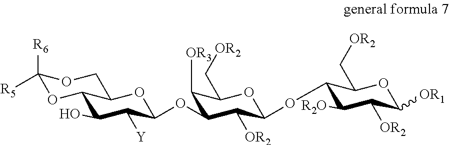

wherein R₁ is a group removable by catalytic hydrogenolysis,
R₂ is optionally substituted acyl,
R₃ is H or optionally substituted acyl,
R₅ is alkyl or optionally substituted phenyl,
R₆ is H, alkyl or optionally substituted phenyl, or
R₅ and R₆ with the carbon atom to which they are attached form a C₃-C₆ cycloalkyl ring, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

According to an embodiment of the first aspect, a compound of general formula 7 is obtained in the reaction of a compound of general formula 9

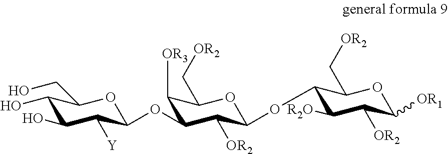

wherein R₁ is a group removable by catalytic hydrogenolysis,
R₂ is optionally substituted acyl,
R₃ is H or optionally substituted acyl, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
   with an aldehyde or ketone of formula R₅R₆C═O or di-O-alkyl-acetal/ketal thereof,
wherein R₅ is selected from alkyl or optionally substituted phenyl,
R₆ is selected from H, alkyl or optionally substituted phenyl, and
R₅ and R₆ with the carbon atom to which they are attached form a C₃-C₆ cycloalkyl ring.

According to an embodiment of the first aspect, a compound of general formula 9 is obtained in the reaction of a compound of general formula 12

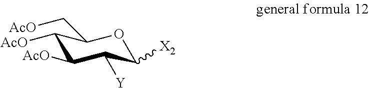

wherein X₂ is selected from halogen, —OC(═NH)CCl₃, —OAc, —OBz or —SR₇, in which R₇ is alkyl or optionally substituted phenyl,
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido, or
Y with the vicinal X₂ forms 2-alkyl-, 2-haloalkyl- or 2-(optionally substituted phenyl)-oxazoline,
   with an acceptor of general formula 11

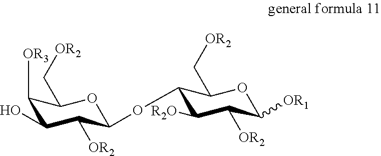

wherein R₁ is a group removable by catalytic hydrogenolysis,
R₂ is optionally substituted acyl,
R₃ is H or optionally substituted acyl.

The second aspect of the invention relates to a compound of general formula 1

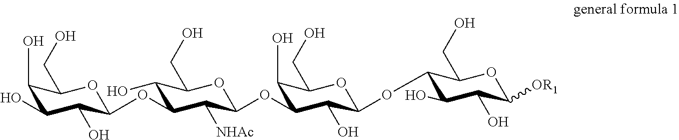

wherein R₁ is a group removable by catalytic hydrogenolysis,
   in crystalline form.

The third aspect of the invention relates to a compound of general formula A

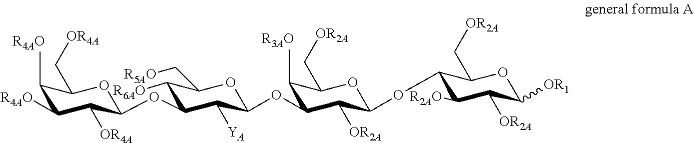

wherein R₁ is a group removable by catalytic hydrogenolysis,
R_{2A} is selected from the group of optionally substituted acyl and H,
R_{3A} is selected from the group of optionally substituted acyl and H,
R_{4A} is selected from the group of optionally substituted acyl and H,
R_{5A} is H,
R_{6A} is H, or
R_{5A} and R_{6A} together form a moiety

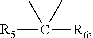

wherein R₅ is alkyl or optionally substituted phenyl, R₆ is H, alkyl or optionally substituted phenyl, or R₅ and R₆ with the carbon atom to which they are attached form a C₃-C₆ cycloalkyl ring, and
Y_{A} is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido, 2,3-dimethylmaleimido and —NH₂,
   provided that if Y_{A} is —NHAc then R_{2A}, R_{3A}, R_{4A}, R_{5A} and R_{6A} cannot be H in the same time.

The fourth aspect of the invention relates to a compound of general formula 7

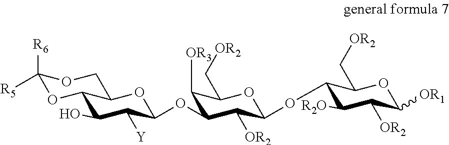

wherein R₁ is a group removable by catalytic hydrogenolysis,
R₂ is optionally substituted acyl,
R₃ is H or optionally substituted acyl,
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
R₅ is alkyl or optionally substituted phenyl,
R₆ is H, alkyl or optionally substituted phenyl, or
R₅ and R₆ with the carbon atom to which they are attached form a C₃-C₆ cycloalkyl ring.

The fifth aspect of the invention relates to a compound of general formula 9

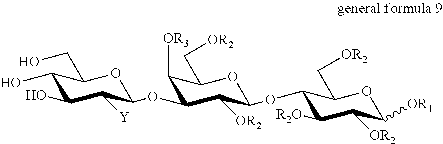

wherein R₁ is a group removable by catalytic hydrogenolysis,
R₂ is optionally substituted acyl,
R₃ is H or optionally substituted acyl, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

The sixth aspect of the invention relates to a compound of general formula 10

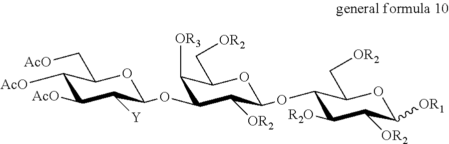

wherein R₁ is a group removable by catalytic hydrogenolysis,
R₂ is optionally substituted acyl,
R₃ is H or optionally substituted acyl,
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

The seventh aspect of the invention relates to the use of a compound of general formula 1 for the preparation of LNT and derivatives thereof, for the production/preparation of human milk oligosaccharides, and for the synthesis of complex oligosaccharides/glycoconjugates suitable for therapeutic/nutritional use.

The eighth aspect of the invention relates to the use of a compound of general formula A for the preparation of LNT and derivatives thereof, for the production/preparation of human milk oligosaccharides, and for the synthesis of complex oligosaccharides/glycoconjugates suitable for therapeutic/nutritional use.

The ninth aspect of the invention relates to the use of a compound of general formula 7 for the preparation of LNT and derivatives thereof, for the production/preparation of human milk oligosaccharides, and for the synthesis of complex oligosaccharides/glycoconjugates suitable for therapeutic/nutritional use.

The tenth aspect of the invention relates to the use of a compound of general formula 9 for the preparation of LNT and derivatives thereof, for the production/preparation of human milk oligosaccharides, and for the synthesis of complex oligosaccharides/glycoconjugates suitable for therapeutic/nutritional use.

The eleventh aspect of the invention relates to the use of a compound of general formula 10 for the preparation of LNT and derivatives thereof, for the production/preparation of human milk oligosaccharides, and for the synthesis of complex oligosaccharides/glycoconjugates suitable for therapeutic/nutritional use.

### DETAILED DESCRIPTION

For a complete understanding of the present invention and the advantages thereof, reference is made to the following detailed description.

It should be appreciated that various embodiments of the present invention can be combined with other embodiments of the invention and are merely illustrative of the specific ways to make and use the invention and do not limit the scope of the invention when taken into consideration with the claims and the following detailed description.

In the present specification, the following features are given a definition that should be taken into account when reading and interpreting the description, examples and claims.

The term “alkyl” refers to a linear or branched hydrocarbon group with 1-6 carbon atoms, such as but not limited to methyl, ethyl, n-propyl, i-propyl, n-butyl, i-butyl, s-butyl, t-butyl, etc.

The term “aryl” refers to homoaromatic groups, such as, but not limited to phenyl or naphthyl.

The term “acyl” refers to a R—C(═O)—, wherein R may be H, alkyl or aryl. Non limiting examples of acyl are formyl, acetyl, propionyl, butyryl, pivaloyl and benzoyl. The term “acyl” in R₂, R_{2A}, R₃, R_{3A}, R₄, R_{4A} and R₇ as carbohydrate protecting groups means C₁-C₆-alkylcarbonyl or arylcarbonyl, like acetyl, pivaloyl, benzoyl, etc.

The term “alkanoylamido” in group Y and Y_{A} refers to C₁-C₆-alkylcarbonyl-NH-group such as, but not limited to, acetamido, propionylamido, etc.

The term “haloalkanoylamido” in group Y and Y_{A} refers to halogen substituted alkanoylamido such as, but limited to, chloroacetamido, trichloroacetamido, trifluoroacetamido, etc.

The term “alkoxycarbonylamino” in group Y and Y_{A} refers to C₁-C₆-alkyloxycarbonyl-NH-group such as, but not limited to, methoxycarbonylamino, ethoxycarbonylamino, etc.

The term “haloalkoxycarbonylamino” in group Y and Y_{A} refers to C₁-C₆-alkyloxycarbonyl-NH-group substituted by one or more halogen atoms such as, but not limited to, 2,2,2-trichloroethoxycarbonylamino, etc.

The term “optionally substituted” refers to a chemical group that may either carry a substituent or may be unsubstituted.

The term “substituted” means that the group in question is substituted with a group which typically modifies the general chemical characteristics of the group in question. The substituents can be used to modify characteristics of the molecule as a whole, such as molecule stability, molecule solubility, and an ability of the molecule to form crystals.

More generally in connection with the terms “alkyl”, “aryl”, “acyl” and “benzamido” the term “optionally substituted” means that the group in question may be substituted one or several times. It is preferable that such groups are optionally substituted 1-5 times, more preferably 1-3 times with group(s) selected from alkyl (only for aryl and aromatic acyl), hydroxy, alkoxy, carboxy, oxo, alkoxycarbonyl, alkylcarbonyl, formyl, aryl, aryloxy-carbonyl, aryloxy, arylamino, arylcarbonyl, amino, mono- and dialkylamino, carbamoyl, mono- and dialkyl-aminocarbonyl, alkylcarbonylamino, cyano, alkanoyloxy, nitro, alkylthio and halogens.

The term “group removable by catalytic hydrogenolysis” refers to groups, whose C—O bond is cleaved by addition of hydrogen in the presence of catalytic amounts of palladium, Raney nickel or another appropriate metal catalyst known for use in hydrogenolysis, resulting in the regeneration of the OH group. Groups of this type are well known to the person skilled in the art and are described for example by P. G. M. Wuts and T. W. Greene: Protective Groups in Organic Synthesis, John Wiley & Sons (2007). Suitable groups include benzyl, diphenylmethyl (benzhydryl), 1-naphthylmethyl, 2-naphthylmethyl or triphenylmethyl (trityl) groups, each of which may be optionally substituted by one or more groups selected from: alkyl, alkoxy, phenyl, amino, acylamino, alkylamino, dialkylamino, nitro, carboxyl, alkoxycarbonyl, carbamoyl, N-alkylcarbamoyl, N,N-dialkylcarbamoyl, azido, halogenalkyl or halogen. Preferably, such substitution, if present, is on the aromatic ring(s). Particularly preferred protecting group is benzyl optionally substituted with one or more groups selected from alkyl or halogen. More preferably, the protecting group is selected from unsubstituted benzyl, 4-chlorobenzyl and 4-methylbenzyl. These particularly preferred and more preferable protecting groups have the advantage that the by-products of the hydrogenolysis are exclusively toluene or substituted toluene. Such toluene or substituted toluene by-products can easily be removed from water soluble oligosaccharide products via evaporation and/or extraction processes.

The present invention provides a method for the large scale manufacture LNT. The method is based upon the introduction of relevant crystalline intermediates permitting simple and robust purification methodologies. Crystallization is one of the simplest and most efficient methods to separate a desired product from contaminants thereby yielding a highly pure desired product. In addition, providing one or more crystalline modifications (polymorphs) of a solid is an important factor in product development, because the different crystalline forms affect the compound's properties—for example thermodynamic stability, solubility, density, hygroscopicity, electrical properties (such as dielectric constant, conductivity), mechanical properties (such as friability, hardness, breaking strength, elasticity), optical properties (such as colour, transparency, refraction), etc.—diversely. It enlarges the repertoire of materials that a scientist has available for improving the product's characteristics.

The method for the manufacture of LNT comprises the step of subjecting a compound of general formula 1

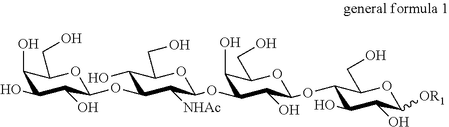

wherein R₁ is a group removable by catalytic hydrogenolysis,
   to catalytic hydrogenolysis.

Removal of the R₁-group by hydrogenolysis typically takes place in a protic solvent or in a mixture of protic solvents. The protic solvent may be selected from a group consisting of water, acetic acid or a C₁-C₆ alcohol. Mixture of one or more protic solvents with one or more proper aprotic organic solvents miscible partially or fully with the protic solvent(s) (such as THF, dioxane, ethyl acetate, acetone, etc.) may also be used. Water, one or more C₁-C₆ alcohols or a mixture of water and one or more C₁-C₆ alcohols are preferably used. Solutions containing the carbohydrate derivatives of general formula 1 in any concentration or suspensions of the carbohydrate derivatives of general formula 1 with the solvent(s) used are also applicable. The reaction mixture is stirred at a temperature of between 10-100° C., preferably between 20-70° C. in a hydrogen atmosphere of 1-50 bar in the presence of a catalyst such as palladium, Raney nickel or any other appropriate metal catalyst, preferably palladium on charcoal or palladium black, until reaching the completion of the reaction. Catalyst metal concentrations generally range from 0.1% to 10% based on the weight of carbohydrate. Preferably, the catalyst concentrations range from 0.15% to 5%, more preferably 0.25% to 2.25%. Transfer hydrogenolysis may also be performed, when the hydrogen is generated in situ from cyclohexene, cyclohexadiene, formic acid or ammonium formate. Addition of organic or inorganic bases/acids and/or basic and/or acidic ion exchange resins can also be used to improve the kinetics of the hydrogenolysis. The use of basic substances is especially preferred when halogen substituents are present on the substituted benzyl moieties of the precursors. Preferred organic bases are including but not limited to triethylamine, diisopropyl ethylamine, ammonia, ammonium carbamate, diethylamine, etc. Preferred organic/inorganic acids include, but are not limited to formic acid, acetic acid, propionic acid, chloroacetic acid, dichloroacetic acid, triflouroacetic acid, HCl, HBr, etc. The conditions above allow simple, convenient and delicate removal of the solvent(s) giving rise to substantially pure LNT. LNT can be isolated from the reaction mixture using conventional work-up procedures in crystalline, amorphous solid, syrupy form or concentrated aqueous solution.

In a preferred embodiment 1-O-benzyl LNT is subjected to catalytic hydrogenolysis to provide the tetrasaccharide LNT. The catalytic hydrogenolysis can be performed in water or in aqueous alcohol, preferably in water, water/methanol or water/ethanol mixture (alcohol content: 10-50 v/v %). The catalytic hydrogenolysis is performed at a temperature of between 15-65° C., preferably between 40-60° C. The catalyst concentration may range from 0.4% to 1.2% (weight of the metal content based on the weight of the carbohydrate of general formula 1).

Both solid forms of LNT such as amorphous/freeze dried/spray dried and liquid forms of LNT such as aqueous solutions/syrups provided by the present invention have high purity suitable for infant nutritional use including but not limited to infant formulas, infant cereals, clinical infant nutritional products. In general, both solid and liquid forms of LNT manufactured according to the present invention are suitable for general nutritional use for infants, toddlers, children, adults and elderly. Both solid and liquid forms of LNT manufactured according to the present invention can also be used as food additives, dietary supplements, a component of alcoholic and non-alcoholic beverages such as, but not limited to soft drinks, fruit juices, bottled water, wine and beer. Both solid and liquid forms LNT manufactured according to the present invention can also be used as a therapeutic agent in broad therapeutic application areas including but not limited to prevent bacterial and viral infections, to avoid diarrhea, to enhance immune system and brain development. Both solid and liquid forms of LNT manufactured according to the present invention can also be used in veterinary applications including but not limited to fight against infectious diseases of domesticated animals. LNT manufactured according to the present invention can also be used as a monomer for the manufacture of polymeric/polymer mounted products providing multivalent binding for bacteria and viruses. LNT manufactured according to the present invention can also be used for the preparation of other human milk oligosaccharides by applying chemical and/or enzymatic methodologies including but not limited to simple structural modifications of further fucosylation, further sialylation, and further extension of the core structure via N-acetyl lactosaminylation/N-acetylisolactosaminylation.

Compounds of general formula 1 can be obtained from compounds of general formula 6

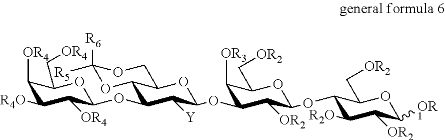

wherein R₁ is as defined above,
R₂ is optionally substituted acyl,
R₃ is H or optionally substituted acyl,
R₄ is optionally substituted acyl,
R₅ is alkyl or optionally substituted phenyl,
R₆ is H, alkyl or optionally substituted phenyl, or
R₅ and R₆ with the carbon atom to which they are attached form a C₃-C₆ cycloalkyl ring, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido, in a one-step reaction or multistep reactions depending on the deprotective conditions used.

In a preferred embodiment, compound of general formula 6, wherein R₂ is optionally substituted acyl, provided that acetyl is excluded, is used for the synthesis of a compound of general formula 1. More preferably, R₃ is H in a compound of general formula 6.

A compound of general formula 5

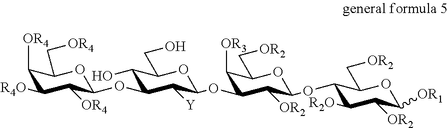

wherein R₁ is a group removable by catalytic hydrogenolysis,
R₂ is optionally substituted acyl,
R₃ is H or optionally substituted acyl,
R₄ is optionally substituted acyl, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
   is obtained when a compound of general formula 6 above is subjected to acid catalysed hydrolysis.

The feature “acid catalysed hydrolysis” refers to a chemical reaction in which water reacts in the presence of acid at pH>2 with a substance bearing acid labile protective group(s) to regenerate the functional group(s) protected. In the present context the acid labile protective groups are protective groups of 1,3-diol systems in the form of cyclic acetals/ketals. In addition, the educt may contain acyl protective groups as well. The skilled person is fully aware that acyl groups can be deprotected by only strong acidic hydrolysis (pH<2). The skilled person is able to distinguish which deprotective condition affects the acetal groups while the acyl groups remain intact. Furthermore the interglycosidic linkages may be also sensitive to acids. The skilled person is fully aware that interglycosidic linkages can be split by only strong acidic hydrolysis (pH<2). The skilled person is able to distinguish which deprotective condition affects the acetal groups while the interglycosidic linkages remain intact. Water—which has to be present in the reaction milieu as reagent—may serve as solvent or co-solvent as well. Organic protic or aprotic solvents which are stable under acidic conditions and miscible fully or partially with water such as C₁-C₆ alcohols, acetone, THF, dioxane, ethyl acetate, MeCN, etc. may be used in a mixture with water. The acids used are generally protic acids selected from but not limited to acetic acid, trifluoroacetic acid, HCl, formic acid, sulphuric acid, perchloric acid, oxalic acid, p-toluenesulfonic acid, benzenesulfonic acid, cation exchange resins, etc., which may be present in from catalytic amount to large excess. The hydrolysis may be conducted at temperatures between 0° C. and reflux until reaching completion which takes from about 2 hours to 3 days depending on temperature, concentration and pH. Preferably, organic acids including but not limited to aqueous solutions of acetic acid, formic acid, chloroacetic acid, oxalic acid, etc. and inorganic acids like hydrochloric acid, perchloric acid, etc. are used. Alternatively, anhydrous C₁-C₆ alcohol including but not limited to methanol, ethanol, propanol, butanol, etc. can also be used for the cleavage of the cyclic acetal/ketal moieties via acid catalysed trans-acetalization/trans-ketalization processes. Catalytic amount of hydrogen chloride, sulphuric acid, perchloric acid, p-toluenesulfonic acid, acetic acid, oxalic acid, champhorsulfonic acid, strong acidic ion-exchange resins, etc. can be used for the purposes at temperatures of 20° C. to reflux.

Preferably, a compound of general formula 6, wherein R₂ is optionally substituted acyl, provided that acetyl is excluded, is used in the acidic deprotection step to obtain a compound of general formula 5. More preferably, R₃ is H in a compound of general formula 6.

Even more preferably a compound of general formula 6, wherein R₅ is optionally substituted phenyl and R₆ is H, more preferably R₅ is phenyl or 4-chlorophenyl, is treated with aqueous acid, preferably perchloric acid solution, to obtain a compound of general formula 5.

Compounds of general formula 1 above can be obtained from compounds of general formula 5

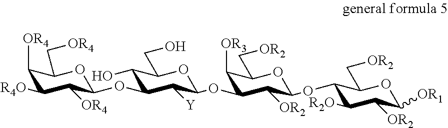

wherein R₁ is a group removable by catalytic hydrogenolysis,
R₂ is optionally substituted acyl,
R₃ is H or optionally substituted acyl,
R₄ is optionally substituted acyl, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
   in a one-step or multistep reaction depending on the deprotective conditions used. Preferably, a compound of general formula 5, wherein R₂ is optionally substituted acyl, provided that acetyl is excluded, is used for the synthesis of a compound of general formula 1. More preferably, R₃ is H in a compound of general formula 5.

Thus a compound of general formula 5 wherein R₁, R₂, R₃ and R₄ are as defined above and Y is —NHAc or —NAc₂, is subjected to base catalysed transesterification reaction or basic hydrolysis to obtain a compound of general formula 1. The feature “base catalysed transesterification reaction or deprotection” means a reaction, where the acyl protective groups from hydroxyls are removed in an alcohol solvent such as methanol, ethanol, propanol, t-butanol, etc. in the presence of an alcoholate, such as, but not limited to NaOMe, NaOEt, KO^{t}Bu, at a temperature of between 20-100° C. The alcohol solvent and the alcoholate should be matched that is to say that ethanol solvent should be used with NaOEt alcoholate. Furthermore a use of a co-solvent as toluene or xylene is beneficial in order to control particle size of the product of general formula 1 and to avoid gel formations. Under this condition only O-acyls can be deprotected and one of the acetyl groups of the —NAc₂ residue is also removed to give a compound having a —NHAc substituent. The alkanoylamido, haloalkanoylamido, carbamate, benzamido and cyclic imide protective groups remain intact under the condition of base catalysed transesterification deprotection. In a preferred embodiment catalytic amount of NaOMe is used in methanol (Zemplén de-O-acylation).

In a preferred embodiment a compound of general formula 5, wherein R₁ is benzyl and Y is acetamido, is subjected to base catalysed transesterification reaction or basic hydrolysis to manufacture compounds of the general formula 1. More preferably, a compound of general formula 5, wherein R₂ is optionally substituted acyl, provided that acetyl is excluded, is used for the synthesis of a compound of general formula 1. Even more preferably, R₃ is H in a compound of general formula 5.

In a multistep sequence, a compound of general formula 5 is transformed into a compound of general formula 1 comprising the steps:
a) conversion of a compound of general formula 5

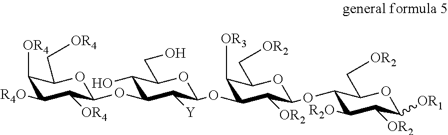

wherein R₁, R₂, R₃ and R₄ are as defined above, and
Y is selected from alkanoylamido, haloalkanoylamido, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
into a compound of general formula 3

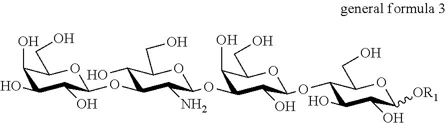

wherein R₁ is as defined above, and
b) conversion of a compound of general formula 3 into a compound of general formula 1.

In a preferred embodiment a compound of general formula 5, wherein R₂ is optionally substituted acyl, provided that acetyl is excluded, is used for the synthesis of a compound of general formula 1. More preferably, R₃ is H in a compound of general formula 5.

With regard to step a) a protected LNT derivative according to general formula 5, wherein R₁, R₂, R₃ and R₄ are as defined above, and Y is selected from alkanoylamido (with the proviso that acetamido is excluded), haloalkanoylamido, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido, is subjected to base catalysed transesterification reaction to give a compound of general formula 4

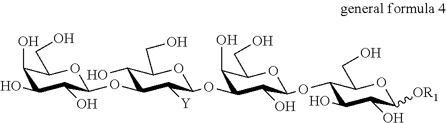

wherein R₁ and Y are as defined above, which compound of general formula 4 is subjected to basic hydrolysis (when Y is selected from haloalkanoylamido, 2,3-diphenylmaleimido and 2,3-dimethylmaleimido), or aminolysis (when Y is selected from alkanoylamido [with the proviso that acetamido is excluded], haloalkanoylamido, alkoxycarbonylamino, haloalkoxycarbonylamino, benzyloxycarbonylamino, optionally substituted benzamido, phthalimido, tetrachlorophthalimido, 2,3-diphenylmaleimido and 2,3-dimethylmaleimido), treatment with Zn (when Y is 2,2,2-trichloroethoxycarbonylamino), catalytic hydrogenolysis (when Y is benzyloxycarbonylamino or azido), or reduction using complex metal hydrides like NaBH₄, or reduction by PPh₃ (when Y is azido) to obtain a compound of general formula 3.

The term “aminolysis” or N-acyl transfer based deprotection means a treatment with ammonia, hydrazine, substituted hydrazine, ethylene diamine or primary amines in water, alcohol or water-organic solvent mixtures at 20-120° C. temperatures. Under this condition all of the O- and N-protecting acyl groups, including carbamates and cyclic imides, can be readily removed.

Trichloroethoxycarbonyl group can be selectively removed via a reductive elimination process with Zn, where Zn can be in the form of Zn dust or in pair with other metal such as Zn-Cu, Zn—Pb, in the presence of acetic acid or ammonium acetate.

Benzyloxycarbonylamino and azido groups can be easily transformed in amino using catalytic hydrogenolysis. It has to be emphasized that these groups are much more reactive under hydrogenolysis conditions than —OR₁ group in compounds of formula 5. The skilled person is aware of the different kinetic behaviour of these groups and able to drive the reaction to reduce benzyloxycarbonylamino and azido to amino without affecting —OR₁ group, for example to run the reaction for shorter time and to stop the reduction before —OR₁ group tends to be split. Alternatively, the azido group can be easily reduced to amino by complex metal hydrides like NaBH₄, or by PPh₃.

In a preferred embodiment a compound of general formula 5, wherein R₁ is benzyl and Y is trichloroacetamido is deprotected under Zemplén condition to give rise to the corresponding compound of general formula 4 (wherein R₁ is benzyl and Y is trichloroacetamido), which is then treated with aqueous base solution to deprotect the amino function to get a compound of general formula 3, wherein R₁ is benzyl.

According to another method in step a), a compound of general formula 5, wherein R₁, R₂, R₃ and R₄ are as defined above, and Y is selected from haloalkanoylamido, 2,3-diphenylmaleimido and 2,3-dimethylmaleimido, can be directly transformed into a compound of general formula 3 by means of basic hydrolysis.

According to a further method in step a), a compound of general formula 5, wherein R₁, R₂, R₃ and R₄ are as defined above, and Y is selected from alkanoylamido, haloalkanoylamido, alkoxycarbonylamino, haloalkoxycarbonylamino, benzyloxycarbonylamino, optionally substituted benzamido, —NAc₂, phthalimido, tetrachlorophthalimido, 2,3-diphenylmaleimido and 2,3-dimethylmaleimido, can be directly transformed into a compound of general formula 3 by means of aminolysis.

The conversion of a compound of general formula 3 into a compound of general formula 1 according to step b) can be realized with selective N-acylation. Selective N-acetylation in the presence of one or more hydroxyls is a well-known reaction and performing such reaction is known to the skilled person. The selective N-acylation involves reaction of the amine of the compound of general formula 3 with a slight excess of acetic anhydride or acetyl chloride (≈1.5-3 equiv.) at a temperature of between approximately 0-35° C. with or without added base. The eventually formed overacetylated by-product(s) can be readily transformed into the desired compounds of general formula 1 with e.g. NaOH/MeOH or NaOMe/MeOH treatment. In another method, derivatives according to general formula 3 are peracetylated, that is the free amino group and all the free hydroxyl groups are acetylated. The compound is treated with acetic anhydride or acetyl chloride, preferably acetic anhydride, in the presence of a base, preferably pyridine, triethylamine or Hünig's base, to give a group of fully protected tetrasaccharides of general formula 2

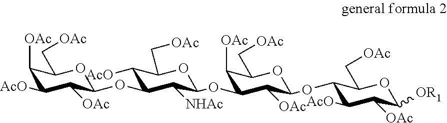

wherein R₁ is defined as above. The peracetylated derivative 2 is then subjected to base catalysed transesterification deprotection or basic hydrolysis (vide supra), preferably to Zemplën de-O-acetylation, to give rise to a compound of general formula 1.

In a preferred embodiment a compound of general formula 3, wherein R₁ is benzyl, is N-acetylated with acetic anhydride (not more than 1.5 equiv) in the presence of aq. NaOH.

According to another embodiment of transforming a compound of general formula 6 to a compound of general formula 1 via multistep deprotection sequence, a compound of general formula 6, wherein R₁, R₂, R₃, R₄, R₅, and R₆ are as defined above, and Y means benzyloxycarbonylamino or azido, is subjected to catalytic hydrogenolysis, as described above, to convert group Y to amino group resulting in a compound of general formula 16

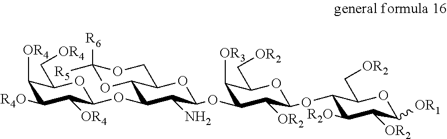

wherein R₁, R₂, R₃, R₄, R₅, and R₆ are as defined above. Preferably R₂ is optionally substituted acyl, provided that acetyl is excluded, and R₃ is H. Alternatively, the azido group can be easily reduced to amino by complex metal hydrides like NaBH₄, or by PPh₃. A compound of general formula 16 so obtained can be de-O-acylated by means of base catalysed transesterification reaction, basic hydrolysis or aminolysis, the conditions of which are disclosed in details above, to give a compound of general formula 14

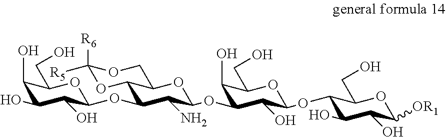

wherein R₁, R₅ and R₆ are as defined above. This compound of general formula 14 is then subjected to acid catalysed hydrolysis (vide supra) to get a compound of general formula 3.

Alternatively, a compound of general formula 16 defined above can be treated with acid to remove the cyclic acetal/ketal protective group to make a compound of general formula 15

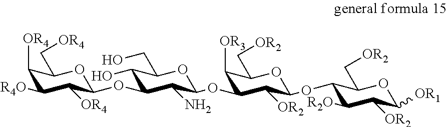

wherein R₁, R₂, R₃ and R₄ are as defined above. Preferably R₂ is optionally substituted acyl, provided that acetyl is excluded, and R₃ is H. The same compound can be synthesized from a compound of general formula 5, wherein Y is benzyloxycarbonyl, azido or 2,2,2-trichloroethoxycarbonylamino, under the conditions described above to convert these functional groups to amino. A compound of general formula 15 is then de-O-acylated using base catalyzed transesterification reaction, basic hydrolysis or aminolysis to give rise to a compound of general formula 3.

According to an alternative way from a compound of general formula 6 to a compound of general formula 14, the starting material, wherein Y means haloalkanoylamido, 2,3-diphenylmaleimido or 2,3-dimethylmaleimido, is subjected to basic hydrolysis. Preferably R₂ is optionally substituted acyl, provided that acetyl is excluded, and R₃ is H. If in compounds of general formula 6 Y is selected from alkanoylamido, haloalkanoylamido, alkoxycarbonylamino, haloalkoxycarbonylamino, benzyloxycarbonylamino, optionally substituted benzamido, —NAc₂, phthalimido, tetrachlorophthalimido, 2,3-diphenylmaleimido and 2,3-dimethylmaleimido, aminolysis also directly leads to compounds of general formula 14.

According to another deprotection pathway, a compound of general formula 6, wherein Y is selected from alkanoylamido, —NAc₂, haloalkanoylamido, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido, is subjected to based catalyzed transesterification reaction to give a compound of general formula 13

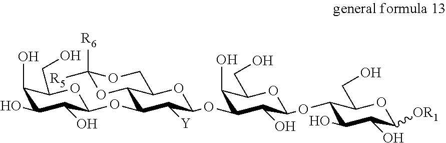

wherein Y means alkanoylamido, haloalkanoylamido, alkoxycarbonylamino, haloalkoxycarbonylamino, benzyloxycarbonylamino, azido, optionally substituted benzamido, phthalimido, tetrachlorophthalimido, 2,3-diphenylmaleimido or 2,3-dimethylmaleimido, which compound of general formula 13 can be converted to a compound of general formula 14 defined above upon basic hydrolysis (if Y means haloalkanoylamido, 2,3-diphenylmaleimido or 2,3-dimethylmaleimido), aminolysis (if Y is selected from alkanoylamido, haloalkanoylamido, alkoxycarbonylamino, haloalkoxycarbonylamino, benzyloxycarbonylamino, optionally substituted benzamido, phthalimido, tetrachlorophthalimido, 2,3-diphenylmaleimido and 2,3-dimethylmaleimido), catalytic hydrogenolysis (if Y means benzyloxycarbonylamino or azido), reduction by complex metal hydrides like NaBH₄, or by PPh₃ (if Y is azido), or Zn/HCl treatment (if Y is 2,2,2-tricholoethoxycarbonylamino). Preferably R₂ is optionally substituted acyl, provided that acetyl is excluded, and R₃ is H.

On the other hand, if any of the compounds of general formula 13 is subjected to acidic hydrolysis, compounds of general formula 4 defined above can be obtained, except for if Y is —NHAc, when a compound of general formula 1 can be made.

A compound of general formula 5 can be converted into a compound of general formula 15 in catalytic hydrogenolysis (if Y means benzyloxycarbonylamino or azido), by reduction by complex metal hydrides like NaBH₄, or by PPh₃ (if Y is azido), or by Zn treatment (if Y is 2,2,2-tricholoethoxycarbonylamino). Preferably R₂ is optionally substituted acyl, provided that acetyl is excluded, and R₃ is H.

In a special case, a compound of general formula 15 wherein R₂ and R₄ is acetyl, and R₃ is acetyl or H, can serve as direct precursor of making a compound of general formula 2 upon acetylation.

A compound of general formula 6, which is a fully protected LNT-derivate and serves as starting material for different deprotection methodologies set forth above, is synthesized in the reaction of a compound of general formula 8 (donor)

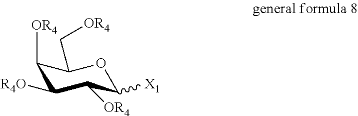

wherein R₄ is optionally substituted acyl, and
X₁ is selected from halogen, —OC(═NH)CCl₃, —OAc, —OBz and —SR₇, wherein R₇ is selected from alkyl and optionally substituted phenyl,
   with a compound of general formula 7 (acceptor)

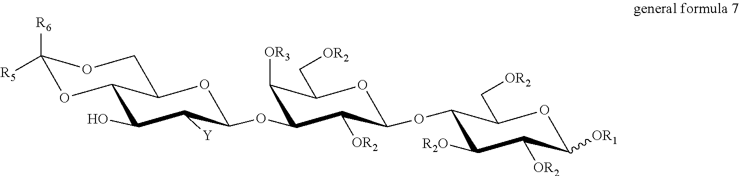

wherein R₁ is a group removable by catalytic hydrogenolysis,
R₂ is optionally substituted acyl,
R₃ is H or optionally substituted acyl,
R₄ is optionally substituted acyl, and
R₅ is selected from alkyl or optionally substituted phenyl,
R₆ is selected from H, alkyl or optionally substituted phenyl, and
R₅ and R₆ with the carbon atom to which they are attached form a C₃-C₆ cycloalkyl ring,
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
   under glycosylation conditions. Preferably R₂ is optionally substituted acyl, provided that acetyl is excluded, and more preferably R₃ is H.

The coupling of the trisaccharide acceptor of general formula 7 with the galactosyl donor of general formula 8 can be carried out an aprotic solvent or in a mixture of aprotic solvents in the presence of an activator (promoter or catalyst) so as to lead to the desired galactosylated product. The new interglycosidic linkage is formed by the nucleophilic displacement of the leaving group X₁ of donor according to general formula 8 with the 3″-OH group of the acceptor according to general formula 7. Other functional groups in both participating reactants have to be masked with protecting groups. In some cases less reactive or hindered OH-group of acceptor is not needed to be blocked (e.g. R₃). Particular care has to be taken with regard to the stereoselectivity. The stereochemical outcome may be affected by different factors like the presence or absence of a participating group at C-2 of the donor, the nature of the leaving group X₁, solvent effect, nature of the protective groups on both the donor and acceptor, nature of the promoters or catalysts, temperature, pressure, steric interactions between the donor and acceptor, and like. In case of galactosaminyl derivatives an array of anomeric activation for glycosylation has been developed and is available to a skilled person engaged in synthetic carbohydrate chemistry.

The glycosyl halides (X₁ means F, Cl, Br, I) are frequently used in glycosylation reaction because of their easy accessibility and satisfactory reactivity. Typically, anomeric halides follow the reactivity order F<Cl<Br<I for nucleophilic displacement. The glycosylation reactions are generally promoted by heavy metal ion, mainly mercury or silver, and Lewis acids.

In a typical glycosidation reaction of glycosyl trichloroacetimidates (X₁═—OC(═NH)CCl₃) catalytic amount of Lewis acid, such as trimethylsilyl triflate or BF₃-etherate, promotes the coupling.

Glycosyl acetates or benzoates (X₁ represents —OAc or —OBz) in glycosylation reaction are first subjected to electrophilic activation providing a reactive intermediate, then treated with the nucleophilic OH-acceptor. Typical activators of choice are Bronsted acids (such as TsOH, HClO₄, sulfamic acid), Lewis acids (such as ZnCl₂, SnCl₄, triflate salts, BF₃-etherate, trityl perchlorate, AlCl₃, triflic anhydride) and their mixtures.

Pentenyl glycosides (X₁ means —O—(CH₂)₃—CH═CH₂) as glycosyl donors can be transglycosylated with appropriate glycosyl acceptors in the presence of a promoter such as NBS and NIS. Protic or Lewis acids (triflic acid, Ag-triflate, etc.) may enhance the reaction.

Thioglycosides (X₁ denotes alkylthio- or phenylthio-group) can be activated by thiofilic promoters such as mercury(II) salts, Br₂, I₂, NBS, NIS, triflic acid, triflate salts, BF₃-etherate, trimethylsilyl triflate, dimethyl-methlythio sulphonium triflate, phenylselenyl triflate, iodonium dicollidine perchlorate, tetrabutylammonium iodide or mixtures thereof, in condensation reactions, preferably by Br₂, NBS, NIS and triflate salts.

Galactosyl donors of general formula 8 can be easily prepared by known methods. Glycosyl iodides, bromides and chlorides (X₁═I, Br, Cl) can be synthesized by treatment of available peracylated galactose with appropriate halogenating agent (e.g. hexamethyl-disilazane/I₂, trimethyl iodosilane, Et₃SiH/I₂, HBr, PBr₃, thionyl chloride, PCl₅/BF₃-etherate, TiCl₄, etc.). The glycosyl fluorides (X₁═F) may be prepared by treatment of the appropriate precursors such as hemiacetals, glycosyl halides (I, Br, Cl), glycosyl esters and S-glycosides with fluorinating reagents such as HF, AgF, AgBF₄, tetrabutyl ammonium fluoride, diethylaminosulfur trifluoride, 2-fluoro-1-methylpyridinium tosylate, Selectfluor, Deoxo-Fluor, 4-methyl(difluoroiodo)benzene, etc. Trichloroacetimidates (X₁═—OC(═NH)CCl₃) can be easily obtained by the addition of the free anomeric OH of the protected hemiacetal to trichloroacetonitrile under inorganic or organic base catalysis. The pentenyl glycosides (X₁ means —O—(CH₂)₃—CH═CH₂) can be prepared with the aid of n-pentenol by standard Fischer glycosylation of hemiacetals under acidic condition, by silver(I) salt promoted coupling of glycosyl bromides (Koenigs-Knorr method), or by glycosylation of 1-acetyl glycosides in the presence of tin(IV) chloride. Thioglycosides (X₁═—SR₇, in which R₇ is alkyl or optionally substituted phenyl) can be achieved by thiolysis of peracylated galactose with R₇SH in the presence of a Lewis acid.

In a preferred embodiment the glycosyl donor is a compound of general formula 8, wherein R₄ is optionally substituted acyl, and X₁ is —SR₇, wherein R₇ is alkyl or optionally substituted phenyl; more preferably R₇ is optionally substituted phenyl; even more preferably R₄ is acetyl, R₇ is phenyl and —SR₇ is in β. The glycosylation is carried out in aprotic solvent(s) like chloroform, dichloromethane, toluene, dioxane, THF, acetonitrile or mixture thereof, preferably chloroform or dichloromethane, under the activation of NIS, NBS, Br₂, triflic acid, silver triflate, BF₃-etherate or mixture thereof. Also preferably, R₂ group in acceptor of general formula 7 means optionally substituted acyl provided that acetyl is excluded, and more preferably R₃ is H.

A compound of general formula 7 ready for glycosylation is available from a compound of general formula 9

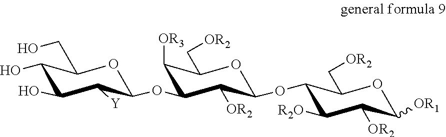

wherein R₁ is a group removable by catalytic hydrogenolysis,
R₂ is optionally substituted acyl,
R₃ is H or optionally substituted acyl,
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
   when treated with an aldehyde or ketone of formula R₅R₆C═O or di-O-alkyl-acetal/ketal thereof, wherein R₅ is selected from alkyl or optionally substituted phenyl, R₆ is selected from H, alkyl or optionally substituted phenyl, and R₅ and R₆ with the carbon atom to which they are attached form a C₃-C₆ cycloalkyl ring, under acidic activation.

The cyclic acetal/ketal formation typically takes place in aprotic solvent or mixture thereof. The reagent aldehyde/ketone or di-O-alkyl-acetal/ketal thereof—if that is liquid—may also serve as solvent. The acids used for promoting acetal/ketal formation (in case of R₅R₆C═O) or transacetalation/transketalation (in case of R₅R₆C═O or di-O-alkyl-acetal/ketal) are generally protic organic (benzenesulfonic acids, camphorsulfonic acid, etc.) and inorganic acids (HCl, HBr, sulfuric acid, perchloric acid, etc.) and Lewis acids (ZnCl₂, FeCl₃, SnCl₂, CuSO₄, AlCl₃, BF₃-etherate, etc.). Continuous extraction of water and/or alcohol formed as by-product during the reaction by means of e.g. scavenger or distillation can be beneficial for the product formation.

In a preferred aspect benzaldehyde, substituted benzaldehyde or di-O-acetals thereof are employed with the aid of benzenesulfonic acids (PhSO₃, 4-Me-PhSO₃) for 4″,6″-acetal formation. Typically aprotic solvent such as benzene, toluene, dichloromethane, chloroform, DMF, THF, dioxane, etc., or mixture thereof is the solvent of choice. Also preferably, R₂ group in compound of general formula 9 means optionally substituted acyl provided that acetyl is excluded, and more preferably R₃ is H.

Compound of general formula 9 defined above involves selective acidic deacetylation of a compound of general formula 10

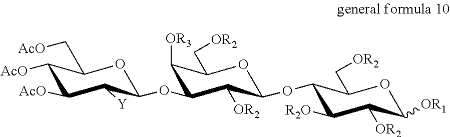

wherein R₁ is a group removable by catalytic hydrogenolysis,
R₂ is optionally substituted acyl,
R₃ is H or optionally substituted acyl,
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

Compounds of general formula 9 can be obtained in a delicate way from compounds of general formula 10. The present inventors recognized that the acetyl groups in glucosaminyl residue can be selectively removed when the lactose portion is protected by acyls different from acetyl due to the higher reactivity of acetyls towards acidic transesterification than other acyls. Acetyl group from the galactose 4-position (R₃ is acetyl) may also be eliminated. The deprotection step can be carried out in a C₁-C₆ alcohol or mixture of C₁-C₆ alcohols, preferably methanol or ethanol in the presence of an acid, generally a protic acid selected from but not limited to acetic acid, trifluoroacetic acid, HCl, formic acid, sulphuric acid, perchloric acid, oxalic acid, p-toluenesulfonic acid, benzenesulfonic acid, cation exchange resins, etc., preferably strong inorganic acid which may be present in from catalytic amount to excess. The use of aprotic co-solvents (dichloromethane, chloroform, dioxane, THF, etc.) may be applicable. The hydrolysis may be conducted at temperatures between 0 and 25° C., preferably at 5-20° C. until TLC shows complete or nearly complete reaction which takes from about 2 hours to 3 days depending on temperature, concentration and pH.

A preferred method encompasses deacetylation of compounds of general formula 10, wherein R₂ means optionally substituted acyl provided that acetyl is excluded, preferably optionally substituted benzoyl, more preferably benzoyl or 4-chlorobenzoyl, and R₃ is H. The reaction is conducted in alcohol, preferably in methanol or ethanol, or in mixture of methanol or ethanol with dichloromethane or THF, in the presence of sulfuric acid, HCl or perchloric acid.

Compounds of general formula 10 defined above is produced in the reaction of donor of general formula 12

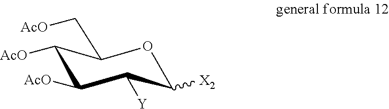

wherein X₂ is selected from halogen, —OC(═NH)CCl₃, —OAc, —OBz or —SR₇, in which
R₇ is alkyl or optionally substituted phenyl,
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido, or
Y with the vicinal X₂ forms 2-alkyl-, 2-haloalkyl- or 2-(optionally substituted phenyl)-oxazoline,
   with acceptor of general formula 11

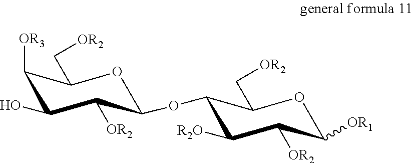

wherein R₁ is a group removable by catalytic hydrogenolysis,
R₂ is optionally substituted acyl,
R₃ is H or optionally substituted acyl,
   under glycosylation conditions. Preferably, R₂ group in compound of general formula 11 means optionally substituted acyl provided that acetyl is excluded, and more preferably R₃ is H.

The coupling of the lactose acceptor of general formula 11 with the glucosaminyl donor of general formula 12 can be carried out an aprotic solvent or in a mixture of aprotic solvents in the presence of an activator (promoter or catalyst) so as to lead to the desired glycosylated product. The new interglycosidic linkage is formed by the nucleophilic displacement of the leaving group X₂ of donor according to general formula 12 with the 3′-OH group of the acceptor according to general formula 11. In addition the present inventors realized that regioselective glycosylation can be achieved on acceptor of general formula 11, wherein R₃ is H. In such dihydroxy acceptors the reactivity of the equatorial 3′-OH and the axial 4′-OH is different: the equatorial OH-group may act as stronger nucleophile under glycosylation conditions. Thus with careful selection of the conditions such as donor reactivity, solvent, temperature, nature of promoter, means of addition of reactants/promoters and like the reaction can be driven to the formation of the desired 1-3 interglycosidic linkage instead of 1-4 coupling. Particular care has to be taken with regard to the stereoselectivity. The stereochemical outcome may be affected by different factors like the presence or absence of a participating group at C-2 of the donor, the nature of the leaving group X₂, solvent effect, nature of the protective groups on both the donor and acceptor, nature of the promoters or catalysts, temperature, pressure, steric interactions between the donor and acceptor, and like. In case of galactosaminyl derivatives an array of anomeric activation for glycosylation has been developed and is available to a skilled person engaged in synthetic carbohydrate chemistry. These methodologies are expansively discussed by reviews and handbooks, for instance by Demchenko (Ed.): Handbook of Chemical Glycosylation, Wiley (2008). For completeness, some general considerations are briefly mentioned below depending on the anomeric substituent (the protecting groups of the acceptors and donors remain intact under glycosylation).

The glycosyl halides (X₂ means F, Cl, Br, I) are frequently used in glycosylation reaction because of their easy accessibility and satisfactory reactivity. Typically, anomeric halides follow the reactivity order F<Cl<Br<I for nucleophilic displacement. The glycosylation reactions are generally promoted by heavy metal ion, mainly mercury or silver, and Lewis acids.

In a typical glycosidation reaction of glycosyl trichloroacetimidates (X₂═—OC(═NH)CCl₃) catalytic amount of Lewis acid, such as trimethylsilyl triflate or BF₃-etherate, promotes the coupling.

Glycosyl acetates or benzoates (X₂ represents —OAc or —OBz) in glycosylation reaction are first subjected to electrophilic activation providing a reactive intermediate, then treated with the nucleophilic OH-acceptor. Typical activators of choice are Bronsted acids (such as TsOH, HClO₄, sulfamic acid), Lewis acids (such as ZnCl₂, SnCl₄, triflate salts, BF₃-etherate, trityl perchlorate, AlCl₃, triflic anhydride) and their mixtures.

Thioglycosides (X₂ denotes alkylthio- or phenylthio-group) can be activated by thiofilic promoters such as mercury(II) salts, Br₂, I₂, NBS, NIS, triflic acid, triflate salts, BF₃-etherate, trimethylsilyl triflate, dimethyl-methlythio sulphonium triflate, phenylselenyl triflate, iodonium dicollidine perchlorate, tetrabutylammonium iodide or mixtures thereof, in condensation reactions, preferably by Br₂, NBS, MS and triflate salts.

Oxazoline derivatives (Y with the vicinal X₂ forms 2-alkyl-, 2-haloalkyl- or 2-(optionally substituted phenyl)-oxazoline) can be promoted in glycosylation reaction with TsOH, camphorsulfonic acid, TMSOTf, FeCl₃, CuCl₂ or pyridinium p-toluenesulfonate.

In a preferred embodiment the glycosyl acceptor is a compound of general formula 11, in which R₁ is optionally substituted benzyl and R₃ is selected from H and optionally substituted benzoyl; more preferably R₁ is benzyl, R₂ is benzoyl optionally substituted with chloro and R₃ is selected from H and benzoyl optionally substituted with chloro, and OR₁ is in β. Preferred glycosyl donors of general formula 12 are those wherein X₂ is —SR₇, in which R₇ is alkyl or optionally substituted phenyl, preferably phenyl and OR₁ is in β, Y is haloalkanoylamido, preferably trichloroacetamido or Y with the vicinal X₂ forms 2-methyl- or 2-trichloromethyl-oxazoline. The glycosylation is preferably conducted in aprotic solvent(s) like chloroform, dichloromethane, toluene, dioxane, THF, acetonitrile or mixture thereof, preferably chloroform or dichloromethane, under the activation of NIS, NBS, Br₂, triflic acid, silver triflate, BF₃-etherate or mixture thereof.

With regard to the synthesis of donors of general formula 12, some literature examples are mentioned below just to illustrate some possible pathways without limitation, the skilled person is capable of combining them to achieve the desired embodiments characterized by general formula 12.

The amino group of glucosamine can be protected with, for instance, acyl, haloacyl (like trichloroacetyl), diacetyl, alkoxycarbonyl, haloalkoxycarbonyl, benzyloxycarbonyl, optionally substituted benzoyl, phthalyl, tetrachlorophthalyl, dimethylmaleolyl or diphenylmaleolyl group. These groups can be introduced in the reaction of the amine with the activated acyl derivatives such as anhydrides, halogenides, active esters, etc. in the presence or absence of a base. The N-protected glucosamine derivatives obtained may be brought to reactions for protecting OH-groups. For instance, peracylation can be conducted with an acylating agent such as halogenides, anhydrides or active derivatives of carboxylic acids (e.g. imidazolide, thioester, silyl ester, vinyl ester, tetrazolide, ortoester, hydroxy-benztriazolyl ester, etc.) in the presence of a base like pyridine, triethylamine, diisopropyl ethylamine, dimethylaminopyridine, etc. in organic solvents such as DCM, chloroform, THF, dioxane, acetonitrile, etc. or mixture thereof at −20-80° C. These peracylated derivatives can also be prepared from glucosamine via peracylation followed by amine protection. Selective removal of the 1-O-acyl group (e.g. with water in the presence of Lewis or Bronsted acid) results in the protected glycosyl hemiacetal which may be converted in a trichloroacetimidate donor with trichloroacetonitrile under inorganic or organic base catalysis. Glycosyl iodides, bromides and chlorides (X₂═I, Br, Cl) can be synthesized by treatment of the 1-O-acyl derivative with appropriate halogenating agent (e.g. hexamethyl-disilazane/I₂, trimethyl iodosilane, Et₃SiH/I₂, HBr, PBr₃, thionyl chloride, PCl₅/BF₃-etherate, TiCl₄, etc.). The glycosyl fluorides (X₂═F) may be prepared by treatment of the appropriate precursors such as hemiacetals, glycosyl halides (I, Br, Cl), glycosyl esters and S-glycosides with fluorinating reagents such as HF, AgF, AgBF₄, tetrabutyl ammonium fluoride, diethylaminosulfur trifluoride, 2-fluoro-1-methylpyridinium tosylate, Selectfluor, Deoxo-Fluor, 4-methyl(difluoroiodo)benzene, etc. Thioglycosides (X₂═—SR₇, in which R₇ is alkyl or optionally substituted phenyl) can be achieved by thiolysis of the 1-O-acyl derivatives or glycosyl halides with R₇SH in the presence of a Lewis acid. Oxazoline-type donors can be synthesized from the appropriate acylamido derivative having any of the X₂ leaving group mentioned above when treated with activators generally used in glycosylations. [azides?]

Compounds of general formula 11 are available by the following manipulations. Starting from the common octa-O-acetyl lactose or hepta-O-acetyl lactosyl bromide the corresponding lactoside can be formed with R₁OH under Lewis-acid (e.g. mercury salt, BF₃-etherate) activation. By de-O-acetylation (e.g. Zemplén-deprotection, aminolysis or basic hydrolysis) followed by regioselective acetonidation with dimethoxypropane in the presence of acid catalyst the 3′,4′-protected lactoside may be obtained, which is then acylated with R₂-halogenide or (R₂)₂O (anhydride) under usual conditions. The resulting derivative may be hydrolysed with acid to remove isopropylidene giving a diol (compounds of general formula 11, wherein R₃ is OH) which is treated with an orthoester derived from R₃OH. A cyclic orthoester thus obtained is subsequently rearranged with acid catalyst to another compound of general formula 11, wherein R₃ is acyl [see e.g. Paulsen et al. Carbohydr. Res. 1985, 137, 39; Lubineau et al. ibid. 1997, 305, 501; and references cited therein] (Scheme 2.).

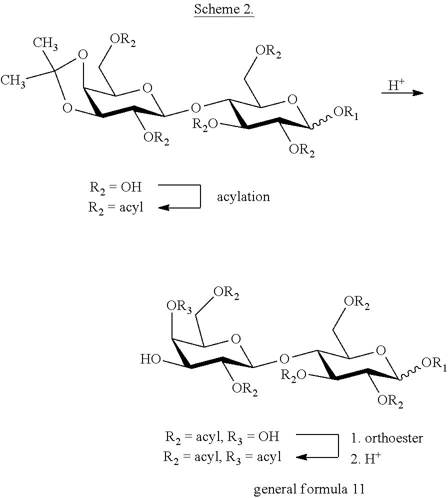

Compounds of general formulae 1, 7, 9, 10 defined above and compounds of general formula A

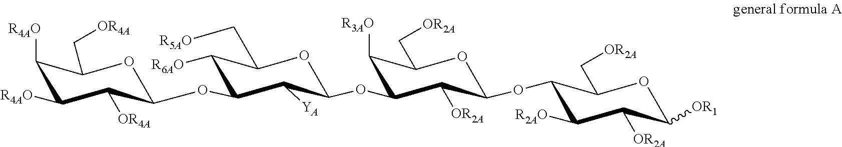

wherein R₁ is a group removable by catalytic hydrogenolysis,
R_{2A} is selected from the group of optionally substituted acyl and H,
R_{3A} is selected from the group of optionally substituted acyl and H,
R_{4A} is selected from the group of optionally substituted acyl and H,
R_{5A} is H,
R_{6A} is H, or
R_{5A} and R_{6A} together form a moiety

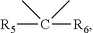

wherein R₅ is alkyl or optionally substituted phenyl, R₆ is H, alkyl or optionally substituted phenyl, or R₅ and R₆ with the carbon atom to which they are attached form a C₃-C₆ cycloalkyl ring, and
Y_{A} is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido, 2,3-dimethylmaleimido and —NH₂,
provided that if Y_{A} is —NHAc then R_{2A}, R_{3A}, R_{4A}, R_{5A} and R_{6A} cannot be H in the same time.
   involving fully or partially protected tetrasaccharides of general formulae 2, 3, 4, 5, 6, 13, 14, 15 and 16 defined above are believed to be valuable synthetic intermediates towards LNT. The present inventors surprisingly recognized some of the compounds of general formulae 1, 7, 9, 10 and A (that is compounds of general formulae 2, 3, 4, 5, 6, 13, 14, 15 and 16) can be obtained in crystalline form. Isolation or purification that uses crystallization makes the whole technological process robust and cost-effective, thus it is advantageous and attractive compared to other procedures.

The present invention has a great commercial value in large scale production of LNT providing high purity of intermediates, which cannot be achieved by any other known purification methods. Although some other intermediates have not shown the ability to crystallize, they can be prepared in clean, high-yielding and less by-product forming reactions where usual work-up (extraction, evaporation, precipitation, etc.) procedures have been sufficient to obtain high purity products which have been used without further purification in the next step.

Thus it is provided valuable LNT intermediates of general formula 1′

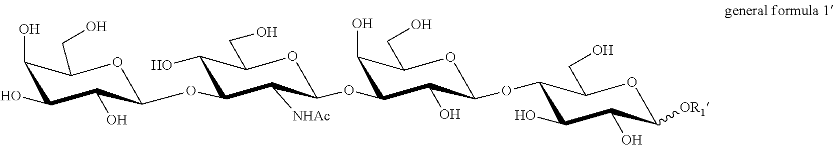

wherein R_{1′} is a group removable by catalytic hydrogenolysis, provided that benzyl is excluded.

In a preferred embodiment R₁ is substituted benzyl, preferably 4-chlorobenzyl or 4-methylbenzyl.

It is strongly emphasised that novel derivatives characterized by general formula 1′ can be considered as sole chemical entities such as either α or β anomers or even an anomeric mixture of α and β isomers, preferably as β-anomer. Novel tetrasaccharide Galpβ1-3GlcNAcpβ1-3Galpβ1-4Glc (lacto-N-tetraose, LNT) intermediates of general formula 1′ can be characterized as oils, syrups, precipitated amorphous material or spray dried products.

Compounds of general formula 1′ provided by the present invention can be used for the preparation of the tetrasaccharide LNT itself and derivatives thereof by using chemical/enzymatic methodologies known in the Art. Compounds of general formulas 1′ can also be used as advanced precursors/intermediates for the production/preparation of numerous human milk oligosaccharides. Novel compounds of general formulas 1′ can also be considered as valuable intermediates for the synthesis of complex oligosaccharides/glycoconjugates suitable for therapeutic/nutritional use.

Once compounds of general formula 1

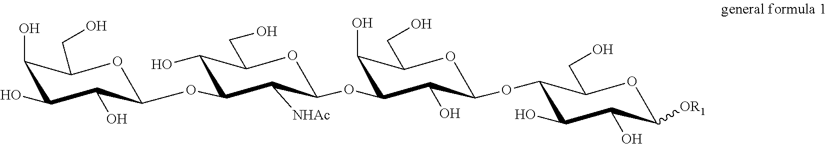

wherein R₁ is a group removable by catalytic hydrogenolysis,
   are prepared by taking whatever route specified above, they can be isolated in crystalline form.

As compounds of general formula 1 are the final intermediates en route to LNT and the last deprotective step runs practically without any by-product formation, their purity is proportional to that of the target product LNT. When crystalline, compounds of general formula 1 might exist either in anhydrous or in hydrated crystalline forms by incorporating one or several molecules of water into their crystal structures. Similarly, novel crystalline compounds characterized by general formula 1 might exist as substances incorporating ligands such as organic molecules and/or ions into their crystal structures.

It is strongly emphasised that novel crystalline derivatives characterized by general formula 1 can be considered as sole chemical entities such as either α or β anomers or even an anomeric mixture of α and β isomers, preferably as β-anomer. Novel crystalline compounds of general formula 1 provided by the present invention can be used for the preparation of LNT itself and derivatives thereof by using chemical/enzymatic methodologies known in the Art. Novel crystalline compounds of general formulas 1 can also be used as advanced precursors/intermediates for the production/preparation of numerous human milk oligosaccharides. Novel crystalline compounds of general formulas 1 can also be considered as valuable intermediates for the synthesis of complex oligosaccharides/glycoconjugates suitable for therapeutic/nutritional use.

In a preferred embodiment R₁ is selected from benzyl, 4-methylbenzyl and 4-chlorobenzyl, preferably benzyl.

The crystallization is carried out from solvent system comprising water miscible solvent. Preferred water miscible solvents including but not limited to are alcohols (methanol, ethanol, propanol, isopropanol, isobutanol, etc., and acetone. More preferably, crystalline compounds of general formula 1 are obtained from aqueous acetone when inducing crystallization.

It is provided valuable LNT intermediates of general formula A

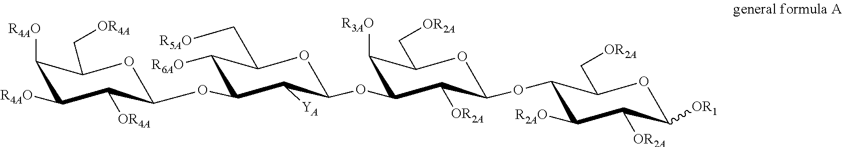

wherein R₁ is a group removable by catalytic hydrogenolysis,
R_{2A} is selected from the group of optionally substituted acyl and H,
R_{3A} is selected from the group of optionally substituted acyl and H,
R_{4A} is selected from the group of optionally substituted acyl and H,
R_{5A} is H,
R_{6A} is H, or
R_{5A} and R_{6A} together form a moiety

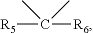

wherein R₅ is alkyl or optionally substituted phenyl, R₆ is H, alkyl or optionally substituted phenyl, or R₅ and R₆ with the carbon atom to which they are attached form a C₃-C₆ cycloalkyl ring, and
Y_{A} is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido, 2,3-dimethylmaleimido and —NH₂,
provided that if Y_{A} is —NHAc then R_{2A}, R_{3A}, R_{4A}, R_{5A} and R_{6A} cannot be H in the same time.
Preferably, R_{2A} group is selected from optionally substituted acyl provided that acetyl is excluded and H, and R_{3A} is H.

It is strongly emphasised that novel derivatives characterized by general formula A can be considered as sole chemical entities such as either α or β anomers or even an anomeric mixture of α and β isomers, preferably as β-anomer. Novel tetrasaccharide LNT intermediates of general formula A can be characterized as crystalline solids, oils, syrups, precipitated amorphous material or spray dried products. If crystalline, compounds of general formula A might exist either in anhydrous or in hydrated crystalline forms by incorporating one or several molecules of water into their crystal structures. Similarly, novel compounds characterized by general formula A might exist as crystalline substances incorporating ligands such as organic molecules and/or ions into their crystal structures.

Novel compounds of general formula A provided by the present invention can be used for the preparation of LNT itself, and other LNT derivatives by using chemical/enzymatic methodologies known in the Art. Novel compounds of general formula A can also be used as advanced precursors/intermediates for the production/preparation of numerous human milk oligosaccharides. Novel compounds of general formulas A can also be considered as valuable intermediates for the synthesis of complex oligosaccharides/glycoconjugates suitable for therapeutic/nutritional use.

As a member of compounds of general formula A it is provided a compound of general formula 2

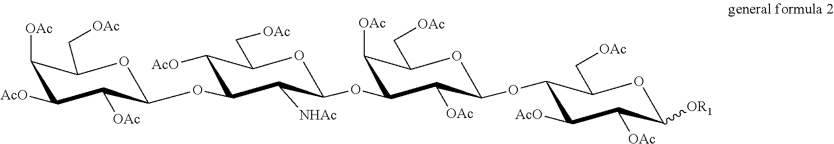

wherein R₁ is a group removable by catalytic hydrogenolysis.

It is strongly emphasised that novel derivatives characterized by general formula 2 can be considered as sole chemical entities such as either α or β anomers or even an anomeric mixture of α and β isomers, preferably as β-anomer. Novel tetrasaccharide LNT intermediates of general formula 2 can be characterized as crystalline solids, oils, syrups, precipitated amorphous material or spray dried products. If crystalline, compounds of general formula 2 might exist either in anhydrous or in hydrated crystalline forms by incorporating one or several molecules of water into their crystal structures. Similarly, novel compounds characterized by general formula 2 might exist as crystalline substances incorporating ligands such as organic molecules and/or ions into their crystal structures.

Novel compounds of general formula 2 provided by the present invention can be used for the preparation of LNT itself, especially when selective N-acetylation of the compounds of general formula 3 is not efficient, and other LNT derivatives by using chemical/enzymatic methodologies known in the Art. Novel compounds of general formulas 2 can also be used as advanced precursors/intermediates for the production/preparation of numerous human milk oligosaccharides. Novel compounds of general formulas 2 can also be considered as valuable intermediates for the synthesis of complex oligosaccharides/glycoconjugates suitable for therapeutic/nutritional use.

In a preferred embodiment R₁ is selected from benzyl, 4-methylbenzyl and 4-chlorobenzyl, preferably benzyl.

Furthermore, as a member of compounds of general formula A, it is provided a compound of general formula 3

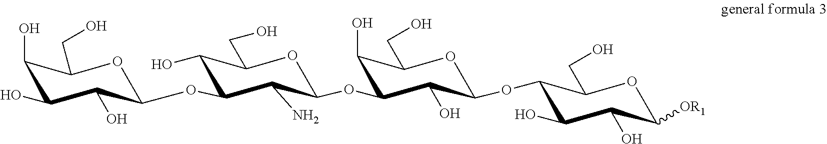

wherein R₁ is a group removable by catalytic hydrogenolysis.

It is strongly emphasised that novel derivatives characterized by general formula 3 can be considered as sole chemical entities such as either α or β anomers or even an anomeric mixture of α and β isomers, preferably as β-anomer. Novel tetrasaccharide LNT intermediates of general formula 3 can be characterized as crystalline solids, oils, syrups, precipitated amorphous material or spray dried products. If crystalline, compounds of general formula 3 might exist either in anhydrous or in hydrated crystalline forms by incorporating one or several molecules of water into their crystal structures. Similarly, novel compounds characterized by general formula 3 might exist as crystalline substances incorporating ligands such as organic molecules and/or ions into their crystal structures.

Novel compounds of general formula 3 provided by the present invention can be used for the preparation of LNT and derivatives thereof by using chemical/enzymatic methodologies known in the Art. Novel compounds of general formulas 3 can also be used as advanced precursors/intermediates for the production/preparation of numerous human milk oligosaccharides. Novel compounds of general formulas 3 can also be considered as valuable intermediates for the synthesis of complex oligosaccharides/glycoconjugates suitable for therapeutic/nutritional use.

In a preferred embodiment R₁ is selected from benzyl, 4-methylbenzyl and 4-chlorobenzyl, preferably benzyl.

Moreover, as a member of compounds of general formula A, it is provided a compound of general formula 4

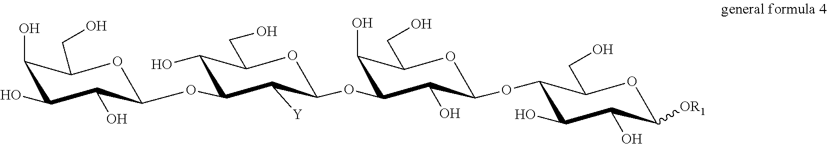

wherein R₁ is a group removable by catalytic hydrogenolysis, and
Y is selected from alkanoylamido, haloalkanoylamido, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

It is strongly emphasised that novel derivatives characterized by general formula 4 can be considered as sole chemical entities such as either α or β anomers or even an anomeric mixture of α and β isomers, preferably as β-anomer. Novel tetrasaccharide LNT intermediates of general formula 4 can be characterized as crystalline solids, oils, syrups, precipitated amorphous material or spray dried products. If crystalline, compounds of general formula 4 might exist either in anhydrous or in hydrated crystalline forms by incorporating one or several molecules of water into their crystal structures. Similarly, novel compounds characterized by general formula 4 might exist as crystalline substances incorporating ligands such as organic molecules and/or ions into their crystal structures.

Novel compounds of general formula 4 provided by the present invention can be used for the preparation of LNT itself and derivatives thereof by using chemical/enzymatic methodologies known in the art. Novel compounds of general formula 4 can also be used as advanced precursors/intermediates for the production/preparation of numerous human milk oligosaccharides. Novel compounds of general formula 4 can also be considered as valuable intermediates for the synthesis of complex oligosaccharides/glycoconjugates suitable for therapeutic/nutritional use.

In a preferred embodiment R₁ is selected from benzyl, 4-methylbenzyl and 4-chlorobenzyl, preferably benzyl, and Y is haloalkanoylamido, preferably trichloroacetamido.

Moreover, as a member of compounds of general formula A, it is provided a compound of general formula 5

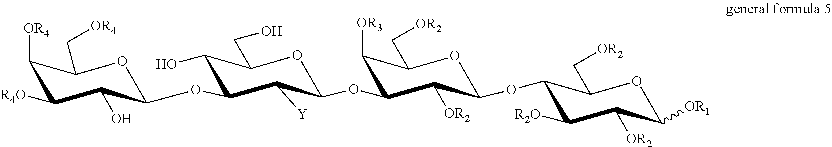

wherein R₁ is a group removable by catalytic hydrogenolysis,
R₂ is optionally substituted acyl,
R₃ is H or optionally substituted acyl,
R₄ is optionally substituted acyl, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

It is strongly emphasised that novel derivatives characterized by general formula 5 can be considered as sole chemical entities such as either α or β anomers or even an anomeric mixture of α and β isomers, preferably as β-anomer. Novel tetrasaccharide LNT intermediates of general formula 5 can be characterized as crystalline solids, oils, syrups, precipitated amorphous material or spray dried products. If crystalline, compounds of general formula 5 might exist either in anhydrous or in hydrated crystalline forms by incorporating one or several molecules of water into their crystal structures. Similarly, novel compounds characterized by general formula 5 might exist as crystalline substances incorporating ligands such as organic molecules and/or ions into their crystal structures.

Novel compounds of general formula 5 provided by the present invention can be used for the preparation of LNT itself or derivatives thereof by using chemical/enzymatic methodologies known in the Art. Novel compounds of general formulas 5 can also be used as advanced precursors/intermediates for the production/preparation of numerous human milk oligosaccharides. Novel compounds of general formulas 5 can also be considered as valuable intermediates for the synthesis of complex oligosaccharides/glycoconjugates suitable for therapeutic/nutritional use.

In a preferred embodiment R₁ is selected from benzyl, 4-methylbenzyl and 4-chlorobenzyl, preferably benzyl, R₂ is optionally substituted acyl provided that acetyl is excluded, preferably optionally substituted benzoyl, more preferably benzoyl or 4-chlorobenzoyl, R₃ is selected from H, acetyl and benzoyl, R₄ is acetyl or benzoyl, and Y is alkanoylamido or haloalkanoylamido, preferably acetamido or trichloroacetamido.

Another aspect of compounds of general formula A relates to the compounds of general formula 6

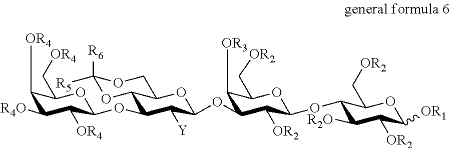

wherein R₁ is a group removable by catalytic hydrogenolysis,
R₂ is optionally substituted acyl,
R₃ is H or optionally substituted acyl,
R₄ is optionally substituted acyl,
R₅ is alkyl or optionally substituted phenyl,
R₆ is H, alkyl or optionally substituted phenyl, or R₅ and R₆ with the carbon atom to which they are attached form a C₃-C₆ cycloalkyl ring, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

It is strongly emphasised that novel derivatives characterized by general formula 6 can be considered as sole chemical entities such as either α or β anomers or even an anomeric mixture of α and β isomers, preferably as β-anomer. Novel tetrasaccharide LNT intermediates of general formula 6 can be characterized as crystalline solids, oils, syrups, precipitated amorphous material or spray dried products. If crystalline, compounds of general formula 6 might exist either in anhydrous or in hydrated crystalline forms by incorporating one or several molecules of water into their crystal structures. Similarly, novel compounds characterized by general formula 6 might exist as crystalline substances incorporating ligands such as organic molecules and/or ions into their crystal structures.

Novel compounds of general formula 6 provided by the present invention can be used for the preparation of LNT itself and derivatives thereof by using chemical/enzymatic methodologies known in the art. Novel compounds of general formulas 6 can also be used as advanced precursors/intermediates for the production/preparation of numerous human milk oligosaccharides. Novel compounds of general formulas 6 can also be considered as valuable intermediates for the synthesis of complex oligosaccharides/glycoconjugates suitable for therapeutic/nutritional use.

In a preferred embodiment R₁ is selected from benzyl, 4-methylbenzyl and 4-chlorobenzyl, preferably benzyl, R₂ is optionally substituted acyl provided that acetyl is excluded, preferably optionally substituted benzoyl, more preferably benzoyl or 4-chlorobenzoyl, R₃ is selected from H, acetyl and benzoyl, but preferably H, R₄ is acetyl or benzoyl, R₅ is optionally substituted phenyl, preferably phenyl or 4-chlorophenyl, R₆ is H, and Y is alkanoylamido or haloalkanoylamido, preferably acetamido or trichloroacetamido.

Moreover, as a member of compounds of general formula A, it is provided a compound of general formula 13

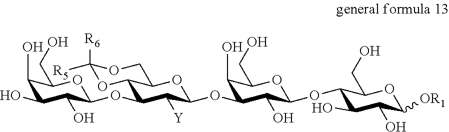

wherein R₁ is a group removable by catalytic hydrogenolysis,
R₅ is alkyl or optionally substituted phenyl,
R₆ is H, alkyl or optionally substituted phenyl, or R₅ and R₆ with the carbon atom to which they are attached form a C₃-C₆ cycloalkyl ring, and
Y is selected from alkanoylamido, haloalkanoylamido, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

It is strongly emphasised that novel derivatives characterized by general formula 13 can be considered as sole chemical entities such as either α or β anomers or even an anomeric mixture of α and β isomers, preferably as β-anomer. Novel tetrasaccharide LNT intermediates of general formula 13 can be characterized as crystalline solids, oils, syrups, precipitated amorphous material or spray dried products. If crystalline, compounds of general formula 13 might exist either in anhydrous or in hydrated crystalline forms by incorporating one or several molecules of water into their crystal structures. Similarly, novel compounds characterized by general formula 13 might exist as crystalline substances incorporating ligands such as organic molecules and/or ions into their crystal structures.

Novel compounds of general formula 13 provided by the present invention can be used for the preparation of LNT itself or derivatives thereof by using chemical/enzymatic methodologies known in the Art. Novel compounds of general formulas 13 can also be used as advanced precursors/intermediates for the production/preparation of numerous human milk oligosaccharides. Novel compounds of general formulas 13 can also be considered as valuable intermediates for the synthesis of complex oligosaccharides/glycoconjugates suitable for therapeutic/nutritional use.

In a preferred embodiment R₁ is selected from benzyl, 4-methylbenzyl and 4-chlorobenzyl, preferably benzyl, R₅ is optionally substituted phenyl, preferably phenyl or 4-chlorophenyl, R₆ is H, and Y is alkanoylamido or haloalkanoylamido, preferably acetamido or trichloroacetamido.

Moreover, as a member of compounds of general formula A, it is provided a compound of general formula 14

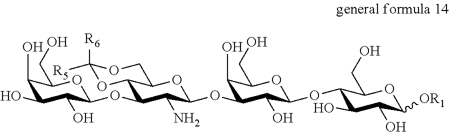

wherein R₁ is a group removable by catalytic hydrogenolysis,
R₅ is alkyl or optionally substituted phenyl,
R₆ is H, alkyl or optionally substituted phenyl,
or R₅ and R₆ with the carbon atom to which they are attached form a C₃-C₆ cycloalkyl ring.

It is strongly emphasised that novel derivatives characterized by general formula 14 can be considered as sole chemical entities such as either α or β anomers or even an anomeric mixture of α and β isomers, preferably as β-anomer. Novel tetrasaccharide LNT intermediates of general formula 14 can be characterized as crystalline solids, oils, syrups, precipitated amorphous material or spray dried products. If crystalline, compounds of general formula 14 might exist either in anhydrous or in hydrated crystalline forms by incorporating one or several molecules of water into their crystal structures. Similarly, novel compounds characterized by general formula 14 might exist as crystalline substances incorporating ligands such as organic molecules and/or ions into their crystal structures.

Novel compounds of general formula 14 provided by the present invention can be used for the preparation of LNT itself or derivatives thereof by using chemical/enzymatic methodologies known in the Art. Novel compounds of general formulas 14 can also be used as advanced precursors/intermediates for the production/preparation of numerous human milk oligosaccharides. Novel compounds of general formulas 14 can also be considered as valuable intermediates for the synthesis of complex oligosaccharides/glycoconjugates suitable for therapeutic/nutritional use.

In a preferred embodiment R₁ is selected from benzyl, 4-methylbenzyl and 4-chlorobenzyl, preferably benzyl, R₅ is optionally substituted phenyl, preferably phenyl or 4-chlorophenyl, and R₆ is H.

Moreover, as a member of compounds of general formula A, it is provided a compound of general formula 15

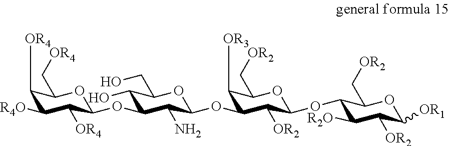

wherein R₁ is a group removable by catalytic hydrogenolysis,
R₂ is optionally substituted acyl,
R₃ is H or optionally substituted acyl,
R₄ is optionally substituted acyl.

It is strongly emphasised that novel derivatives characterized by general formula 15 can be considered as sole chemical entities such as either α or β anomers or even an anomeric mixture of α and β isomers, preferably as β-anomer. Novel tetrasaccharide LNT intermediates of general formula 15 can be characterized as crystalline solids, oils, syrups, precipitated amorphous material or spray dried products. If crystalline, compounds of general formula 15 might exist either in anhydrous or in hydrated crystalline forms by incorporating one or several molecules of water into their crystal structures. Similarly, novel compounds characterized by general formula 15 might exist as crystalline substances incorporating ligands such as organic molecules and/or ions into their crystal structures.

Novel compounds of general formula 15 provided by the present invention can be used for the preparation of LNT itself or derivatives thereof by using chemical/enzymatic methodologies known in the Art. Novel compounds of general formulas 15 can also be used as advanced precursors/intermediates for the production/preparation of numerous human milk oligosaccharides. Novel compounds of general formulas 15 can also be considered as valuable intermediates for the synthesis of complex oligosaccharides/glycoconjugates suitable for therapeutic/nutritional use.

In a preferred embodiment R₁ is selected from benzyl, 4-methylbenzyl and 4-chlorobenzyl, preferably benzyl, R₂ is optionally substituted acyl provided that acetyl is excluded, preferably optionally substituted benzoyl, more preferably benzoyl or 4-chlorobenzoyl, R₃ is selected from H, acetyl and benzoyl, but preferably H, and R₄ is acetyl or benzoyl.

Moreover, as a member of compounds of general formula A, it is provided a compound of general formula 16

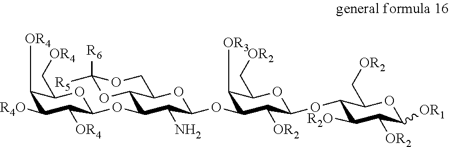

wherein R₁ is a group removable by catalytic hydrogenolysis,
R₂ is optionally substituted acyl,
R₃ is H or optionally substituted acyl,
R₄ is optionally substituted acyl,
R₅ is alkyl or optionally substituted phenyl,
R₆ is H, alkyl or optionally substituted phenyl,
or R₅ and R₆ with the carbon atom to which they are attached form a C₃-C₆ cycloalkyl ring.

It is strongly emphasised that novel derivatives characterized by general formula 16 can be considered as sole chemical entities such as either α or β anomers or even an anomeric mixture of α and β isomers, preferably as β-anomer. Novel tetrasaccharide LNT intermediates of general formula 16 can be characterized as crystalline solids, oils, syrups, precipitated amorphous material or spray dried products. If crystalline, compounds of general formula 16 might exist either in anhydrous or in hydrated crystalline forms by incorporating one or several molecules of water into their crystal structures. Similarly, novel compounds characterized by general formula 16 might exist as crystalline substances incorporating ligands such as organic molecules and/or ions into their crystal structures.

Novel compounds of general formula 16 provided by the present invention can be used for the preparation of LNT itself or derivatives thereof by using chemical/enzymatic methodologies known in the Art. Novel compounds of general formulas 16 can also be used as advanced precursors/intermediates for the production/preparation of numerous human milk oligosaccharides. Novel compounds of general formulas 16 can also be considered as valuable intermediates for the synthesis of complex oligosaccharides/glycoconjugates suitable for therapeutic/nutritional use.

In a preferred embodiment R₁ is selected from benzyl, 4-methylbenzyl and 4-chlorobenzyl, preferably benzyl, R₂ is optionally substituted acyl provided that acetyl is excluded, preferably optionally substituted benzoyl, more preferably benzoyl or 4-chlorobenzoyl, R₃ is selected from H, acetyl and benzoyl, but preferably H, R₄ is acetyl or benzoyl, R₅ is optionally substituted phenyl, preferably phenyl or 4-chlorophenyl, and R₆ is H.

Another aspect of the invention relates to the compounds of general formula 7

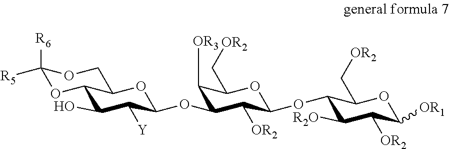

wherein R₁ is a group removable by catalytic hydrogenolysis,
R₂ is optionally substituted acyl,
R₃ is H or optionally substituted acyl,
R₅ is alkyl or optionally substituted phenyl,
R₆ is H, alkyl or optionally substituted phenyl, or R₅ and R₆ with the carbon atom to which they are attached form a C₃-C₆ cycloalkyl ring, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

It is strongly emphasised that novel derivatives characterized by general formula 7 can be considered as sole chemical entities such as either α or β anomers or even an anomeric mixture of α and β isomers, preferably as β-anomer. Novel LNT intermediates of general formula 7 can be characterized as crystalline solids, oils, syrups, precipitated amorphous material or spray dried products. If crystalline, compounds of general formula 7 might exist either in anhydrous or in hydrated crystalline forms by incorporating one or several molecules of water into their crystal structures. Similarly, novel compounds characterized by general formula 7 might exist as crystalline substances incorporating ligands such as organic molecules and/or ions into their crystal structures.

Novel compounds of general formula 7 provided by the present invention can be used for the preparation of lacto-N-tetraose, LNT itself and derivatives thereof by using chemical/enzymatic methodologies known in the Art. Novel compounds of general formulas 7 can also be used as advanced precursors/intermediates for the production/preparation of numerous human milk oligosaccharides. Novel compounds of general formulas 7 can also be considered as valuable intermediates for the synthesis of complex oligosaccharides/glycoconjugates suitable for therapeutic/nutritional use.

In a preferred embodiment R₁ is selected from benzyl, 4-methylbenzyl and 4-chlorobenzyl, preferably benzyl, R₂ is optionally substituted acyl provided that acetyl is excluded, preferably optionally substituted benzoyl, more preferably benzoyl or 4-chlorobenzoyl, R₃ is selected from H, acetyl and benzoyl, preferably H, R₅ is optionally substituted phenyl, preferably phenyl or 4-chlorophenyl, R₆ is H, and Y is alkanoylamido or haloalkanoylamido, preferably acetamido or trichloroacetamido.

Another aspect of the invention relates to the compounds of general formula 9

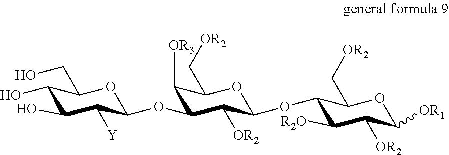

wherein R₁ is a group removable by catalytic hydrogenolysis,
R₂ is optionally substituted acyl,
R₃ is H or optionally substituted acyl, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

It is strongly emphasised that novel derivatives characterized by general formula 9 can be considered as sole chemical entities such as either α or β anomers or even an anomeric mixture of α and β isomers, preferably as β-anomer. Novel LNT intermediates of general formula 9 can be characterized as crystalline solids, oils, syrups, precipitated amorphous material or spray dried products. If crystalline, compounds of general formula 9 might exist either in anhydrous or in hydrated crystalline forms by incorporating one or several molecules of water into their crystal structures. Similarly, novel compounds characterized by general formula 9 might exist as crystalline substances incorporating ligands such as organic molecules and/or ions into their crystal structures.

Novel compounds of general formula 9 provided by the present invention can be used for the preparation of LNT itself and derivatives thereof by using chemical/enzymatic methodologies known in the Art. Novel compounds of general formulas 9 can also be used as advanced precursors/intermediates for the production/preparation of numerous human milk oligosaccharides. Novel compounds of general formulas 9 can also be considered as valuable intermediates for the synthesis of complex oligosaccharides/glycoconjugates suitable for therapeutic/nutritional use.

In a preferred embodiment R₂ is optionally substituted acyl provided that acetyl is excluded. More preferably R₁ is selected from benzyl, 4-methylbenzyl and 4-chlorobenzyl, preferably benzyl, R₂ is optionally substituted benzoyl, preferably benzoyl or 4-chlorobenzoyl, R₃ is selected from H, acetyl and benzoyl, preferably H, and Y is alkanoylamido or haloalkanoylamido, preferably acetamido or trichloroacetamido.

Another aspect of the invention relates to the compounds of general formula 10

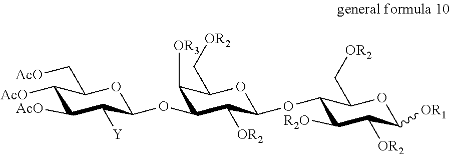

wherein R₁ is a group removable by catalytic hydrogenolysis,
R₂ is optionally substituted acyl,
R₃ is H or optionally substituted acyl, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

It is strongly emphasised that novel derivatives characterized by general formula 10 can be considered as sole chemical entities such as either α or β anomers or even an anomeric mixture of α and β isomers, preferably as β-anomer. Novel LNT intermediates of general formula 10 can be characterized as crystalline solids, oils, syrups, precipitated amorphous material or spray dried products. If crystalline, compounds of general formula 10 might exist either in anhydrous or in hydrated crystalline forms by incorporating one or several molecules of water into their crystal structures. Similarly, novel compounds characterized by general formula 10 might exist as crystalline substances incorporating ligands such as organic molecules and/or ions into their crystal structures.

Novel compounds of general formula 10 provided by the present invention can be used for the preparation of LNT and derivatives thereof by using chemical/enzymatic methodologies known in the Art. Novel compounds of general formulas 10 can also be used as advanced precursors/intermediates for the production/preparation of numerous human milk oligosaccharides. Novel compounds of general formulas 10 can also be considered as valuable intermediates for the synthesis of complex oligosaccharides/glycoconjugates suitable for therapeutic/nutritional use.

In a preferred embodiment R₂ is optionally substituted acyl provided that acetyl is excluded. More preferably R₁ is selected from benzyl, 4-methylbenzyl and 4-chlorobenzyl, preferably benzyl, R₂ is optionally substituted benzoyl, preferably benzoyl or 4-chlorobenzoyl, R₃ is selected from H, acetyl and benzoyl, preferably H, and Y is alkanoylamido or haloalkanoylamido, preferably acetamido or trichloroacetamido.

Other features of the invention will become apparent in the course of the following descriptions of exemplary embodiments which are given for illustration of the invention and are not to be limiting thereof.

### EXAMPLES

### Example 1

### Phenyl 2-deoxy-3,4,6-tri-O-acetyl-2-trichloroacetamido-1-thio-β-D-glucopyranoside

D-Glucosamine hydrochloride (100.0 g, 464.0 mmol) was added to a solution of methanolic NaOMe (300 mL, 2.6 eq. of NaOMe) in MeOH (200 mL) stirred at −5° C. Trichloroacetyl chloride (1.4 eq.) was added dropwise. After 10 min the mixture was carefully neutralized by addition of aq. HCl. The reaction solution was evaporated to ≈300 mL then water (200 mL) was added and the rest of methanol was evaporated. Another portion of water (200 mL) water was added for the crystallization. After one night in the cold room with stirring, the crystals were filtered, washed 2 times with 300 mL iPr₂O and dried overnight to get the N-trichloroacetamide protected compound (95.5 g, 63%) as an off-white solid.

Pyridine (75 mL, 6 eq.) was cooled to 0° C. and 2-deoxy-2-trichloroacetamido-D-glucopyranose (50 g) was added during stirring. Ac₂O (5 eq.) was added dropwise within 3 h. The reaction mixture was stirred 2 h at 0° C. and 12 h at room. The reaction was quenched by dropwise addition of MeOH (7 mL) at 0-5° C. and stirred 30 min followed by dilution with DCM (150 mL). The reaction was washed with water (50 mL), aq. 6M HCl (3×50 mL) and again water (50 mL). The organic extract was washed again with brine (50 mL) and finally with water (50 mL). The DCM was distilled off and the crude peracetate was used directly in the next step.

Thiophenol (20.6 mL, 1.3 eq.) was added to the previous crude material in DCM and the reaction mixture was cooled to 0° C. BF₃Et₂O (28.5 mL, 1.5 eq) was added dropwise in 60 min and the reaction mixture was stirred for 10 h at 45° C. After cooling down to RT, the reaction mixture was washed with water/brine mixture (1:1, 50 mL) followed by aq. 1N NaOH/water mixture (1:1, 2×50 mL). Finally the organic extract was washed with water/brine mixture (1:1, 50 mL). The DCM phase was distilled off and the crude product was crystallized from hexane/ethyl acetate to give the thioglycoside (57.3 g, 105.6 mmol, 69%) as a white solid.

¹H-NMR (CDCl₃, 600 MHz) δ 1.93 (s, 3H, CH₃CO), 2.00 (s, 3H, CH₃CO), 2.10 (s, 3H, CH₃CO), 3.77 (ddd, 1H, J=2.5 5.6 10.1 Hz, H-5), 4.05 (ddd, 1H, J=9.4 9.7 10.1 Hz, H-2), 4.19 (dd, 1H, J=2.5 12.3 Hz, H-6a), 4.23 (dd, 1H, J=5.6 12.3 Hz, H-6b), 4.85 (d, 1H, J=10.1 Hz, H-1), 5.07 (dd, 1H, J=9.6 9.7 Hz, H-3), 5.36 (dd, 1H, J=9.6 10.1 Hz, H-4), 7.05 (d, 1H, J=9.4 Hz, NH), 7.32-7.69 (m, 5H, Ph, NH).

¹³C-NMR (CDCl₃, 150 MHz) δ 20.3 (CH₃CO), 20.5 (CH₃CO), 20.7 (CH₃CO), 54.5 (C-2), 62.3 (C-6), 68.3 (C-3), 73.1 (C-4), 75.9 (C-5), 86.5 (C-1), 92.2 (CCl₃), 128.7 (Ph), 129.0 (Ph), 131.6 (Ph), 133.4 (Ph), 161.7 (COCCl₃), 169.2 (COCH₃), 170.6 (COCH₃), 171.1 (COCH₃).

M.p. 174-175° C.

### Example 2

### Benzyl 2,3,6,2′,6′-penta-O-(4-chlorobenzoyl)-β-D-lactoside

Suspension of 10 g of benzyl β-D-lactoside in acetone (50 ml), dimethoxypropane (3.5 ml) and TMSCl (7 ml) was stirred at rt for 5 h. The mixture was diluted with ethyl acetate (50 ml), filtered, and the cake was washed with ethyl acetate (2×30 ml). The wet cake was dissolved in pyridine (36 ml) and dry DCM (50 ml) and 4-chlorobenzoyl chloride (22 ml) was added slowly to maintain the temperature between 40-45° C. After overnight stirring methanol (10 ml) and DCM (10 ml) were added and extractive work-up was made (2×1M HCl, 1× water, 1× sat. NaHCO₃). The combined organic phase was concentrated and as thick syrup it was poured to 50 ml of isopropanol under intensive stirring. The solid was filtered, washed with isopropanol and dried to result in 20.0 g of benzyl (4-chlorobenzoyl)-3′,4′-di-O-isopropylidene-β-D-lactoside (72%). [α]_{D}=+58.4° (c=1 DCM), Mp: 184° C.

10 g of benzyl 2,3,6,2′,6′-penta-O-(4-chlorobenzoyl)-3′,4′-di-O-isopropylidene-β-D-lactoside was dissolved in DCM (20 ml), acetonitrile (2 ml) and 50% HClO₄ (1 ml) and the mixture was stirred at rt for 30 min. The solution was extracted with sat. NaHCO₃ (2×10 ml), dried, filtered and concentrated. The obtained material was redissolved in ethyl acetate (10 ml) and diluted with hexane (50 ml). The suspension was stirred at rt for 30 min, the filtered to yield 5.4 g of benzyl 2,3,6,2′,6′-penta-O-(4-chlorobenzoyl)-β-D-lactoside as white crystals.

[α]_{D}=+58.65° (c=1 DCM), Mp: 200-201° C.

### Example 3

### Benzyl 4′-O-benzoyl-1,3,6,2′,6′-penta-O-(4-chlorobenzoyl)-β-D-lactoside

To a mixture of 116.6 g of benzyl 2,3,6,2′,6′-penta-O-(4-chlorobenzoyl)-β-D-lactoside in toluene (600 ml) trimethyl orthobenzoate (120 ml) and camphenesulphonic acid (4 g) were added. The mixture was stirred vigorously at rt for 3 h, then 80% acetic acid (160 ml) was added. After further 1 h of stirring the biphasic mixture obtained was separated, the organic phase was diluted with toluene (600 ml), washed with water (800 ml) and sat. NaHCO₃ (2×600 ml), dried, filtered and evaporated. The resulting oil was dropped into 600 ml of heptane and seeded. The white crystalline compound was filtered, washed and dried to yield 110.3 g of product.

[α]_{D}=+17.13° (c=1 DCM), M.p.: 156-157° C.

### Example 4

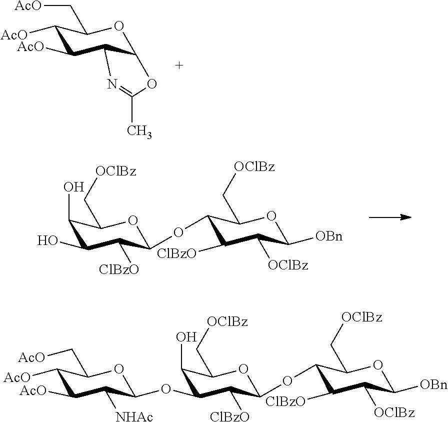

To a solution of 11.12 g (33.8 mmol) of glycosyl donor and 23.74 g (21.0 mmol) of diol acceptor in 400 mL of CH₂Cl₂, 2.6 mL of boron trifluoride diethyl etherate was added and the reaction mixture was refluxed for 2 days, then cooled to room temperature and extracted with sat. NaHCO₃. The separated organic phase was washed with brine, dried on Na₂SO₄, filtered and evaporated to dryness. The crude product was purified by flash chromatography and recrystallized to give 16.8 g of product (55%) as a white solid.

¹³C NMR (500 MHz): 170.0, 169.5, 169.3, 168.6, 164.4, 164.3, 164.1, 163.9, 163.6, 137.1, 131.2, 130.9, 130.8, 129.0, 128.8, 128.7, 128.4, 128.3, 128.1, 128.0, 127.9, 127.7, 127.6, 127.3, 101.3, 100.2, 98.7, 79.8, 75.6, 73.3, 72.5, 72.1, 71.9, 71.7, 70.7, 70.5, 70.3, 68.8, 67.3, 62.9, 62.5, 62.1, 53.7, 21.6, 20.4, 20.3 (two signals).

### Example 5

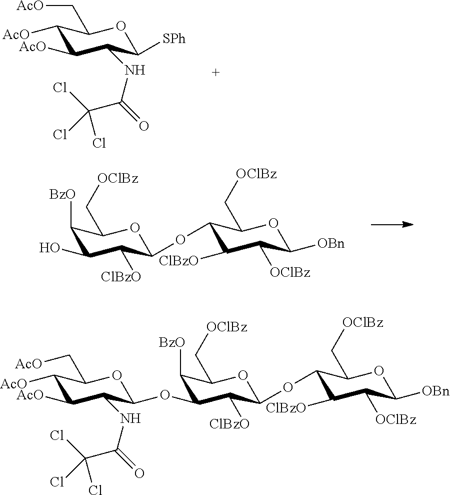

Thiophenyl donor (57 g) and lactose acceptor (100 g) were dissolved in DCM (400 mL) and the solution was cooled to 0-4° C. (ice-bath). After stirring for 15 min, N-bromosuccinimide (20 g) was added. After another 10 min stirring, TfOH (360 μL) was added. The temperature was kept between 0-5° C. for 2 h. The reaction was quenched by dropwise addition of aq. 25% NH₄OH (15.0 mL). The mixture was stirred for 30 min at 0-5° C. Water (100 mL) was added and the two phases were. The organic phase was washed with water (100 mL), water/brine (1/1, 100 mL) and again water/brine (1/1, 100 mL). Half the volume of DCM was evaporated (˜200 mL) and the crude is ready for the next acidic deacetylation without further purification.

¹H-NMR (CDCl₃, 300 MHz) δ 1.87 (s, 3H), 1.93 (s, 3H), 1.96 (s, 3H), 3.38 (dd, 1H, J=7.2 11.4 Hz), 3.45 (ddd, 1H, J=7.8 7.8 10.5 Hz), 3.60 (ddd, 1H, J=3.9 6.6 9.9 Hz), 3.67 (ddd, 1H, J=3.6 3.6 9.6 Hz), 3.72-3.76 (m, 1H), 3.95-4.07 (m, 4H), 4.17 (dd, 1H, J=2.4 12.3 Hz), 4.38-4.40 (m, 2H), 4.55 (d, 1H, J=12.3 Hz), 4.58 (d, 1H, J=7.5 Hz), 4.65 (d, 1H, J=7.8 Hz), 4.80 (d, 1H, J=12.3 Hz), 4.93 (dd, 1H, J=9.9 9.9 Hz), 4.94 (d, 1H, J=7.8 Hz), 5.28 (dd, 1H, J=9.0 10.5 Hz), 5.39 (dd, 1H, J=7.8 9.9 Hz), 5.45 (dd, 1H, J=7.8 10.2 Hz), 5.56 (dd, 1H, J=9.0 9.6 Hz), 5.57 (d, 1H, J=3.3 Hz), 6.41 (d, 1H, J=7.8 Hz), 6.86 (d, 2H, J=8.7 Hz), 7.09-8.00 (m, 28H).

¹³C-NMR (CDCl₃, 75.45 MHz) δ 20.4, 20.5 (2C), 56.6, 61.1, 62.1, 62.7, 68.0, 69.3, 70.1, 70.5, 71.6 (2C), 71.7, 71.8, 72.8, 72.9, 75.3, 76.7, 91.5, 98.7, 99.2, 100.6, 127.1, 127.4, 127.6, 127.7, 127.9, 128.0, 128.3, 128.4, 128.5, 128.7, 129.0-129.1, 130.0, 130.7, 130.8, 130.9, 131.1, 131.3, 133.4, 136.2, 139.4, 139.8, 140.0, 140.1, 140.2, 161.4, 163.6, 164.2, 164.4, 164.9, 165.1, 169.2, 170.1, 170.5.

[α]_{D}²²=+33 (c=1 CHCl₃).

### Example 6

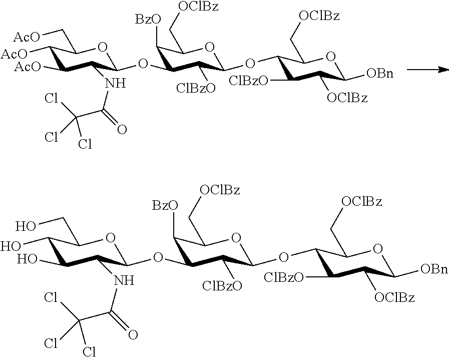

Concentrated sulphuric acid (18 mL) in MeOH 180 (mL) was added to the previous crude trisaccharide in ˜200 mL of DCM. The reaction mixture was stirred at 25° C. for approximately 30 hrs. The mixture is cooled to 5-10° C. and the pH is adjusted to ˜9 by drop wise addition of NH₃ (25% aq.). DCM and MeOH was evaporated at 40° C. and toluene was co-evaporated. The residue was partitioned between water and toluene and then separated. The upper phase was evaporated at 60° C. under reduced pressure and fresh toluene (100 mL) was added. The crude is ready for the next acetal protection.

¹H-NMR (CDCl₃, 300 MHz) δ 2.87-2.97 (m, 1H), 3.13-3.21 (m, 1H), 3.40-4.03 (m, 11H), 4.33-4.42 (m, 2H), 4.55 (d, 1H, J=12.3 Hz), 4.58 (d, 1H, J=7.8 Hz), 4.65 (d, 1H, J=7.8 Hz), 4.80 (d, 1H, J=12.3 Hz), 4.91 (d, 1H, J=8.1 Hz) 5.38 (dd, 1H, J=7.8 9.9 Hz), 5.43 (dd, 1H, J=7.8 10.5 Hz), 5.56 (dd, 1H, J=9.3 9.3 Hz), 5.71 (d, 1H, J=3.3 Hz), 6.85 (d, 2H, J=8.7 Hz), 6.92 (d, 1H, J=6.9 Hz), 7.09-7.95 (m, 28H).

¹³C-NMR (CDCl₃, 75.45 MHz) δ 58.8, 60.8, 61.7, 62.7, 69.5, 70.1, 70.5, 71.3 (2C), 71.4, 71.6, 72.9 (2C), 75.5, 75.8, 77.9, 91.7, 98.7, 99.8, 100.7, 125.3, 127.1, 127.5, 127.6-127.8, 128.1, 128.2, 128.3, 128.4, 128.6, 128.7, 129.0, 129.1, 130.1, 130.8, 130.9, 131.1, 131.4, 134.0, 136.2, 137.8, 139.5, 139.8, 140.0, 140.1, 140.2, 162.1, 163.8, 164.3, 164.5, 164.9, 165.1, 166.4.

[α]_{D}²²=+12.7 (c=1, CHCl₃).

### Example 7

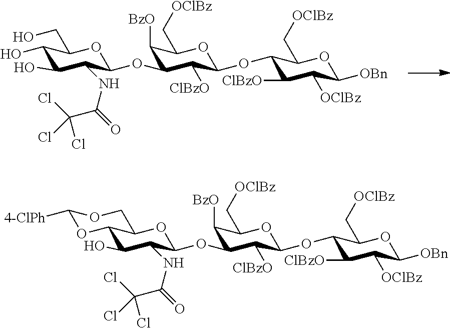

To the previous crude triol-trisaccharide in toluene, 4-chlorobenzaldehyde dimethylacetal (16.0 mL) and p-TsOH×H₂O (1.55 g) were added. The mixture was stirred at 60° C. under reduced pressure with continuous removal of toluene (approx. 150 mL). The reaction was quenched with aq. NH₄OH (3.0 mL, 25%) and tert-butylmethyl ether (360 mL) was added and extracted with water/brine mixture (1:1, 100 mL). The upper phase is the organic phase which was washed another time with water/brine (1:1, 100 mL). The organic solvents were evaporated off under reduced pressure to get an oily residue. The oily residue was crystallized from hexane/i-propyl acetate and dried to get 97.4 g (72%) of product.

¹H-NMR (CDCl₃, 600 MHz) δ 2.71 (d, 1H, J=3.4 Hz, OH), 3.05 (ddd, 1H, J=6.4 8.1 9.9 Hz, H-2″), 3.26 (dd, 1H, J=8.8 8.9 Hz, H-4″), 3.32 (m, 1H, 11-5″), 3.35 (dd, 1H, J=7.8 11.7 Hz, H-6a′), 3.44 (m, 1H, H-6a″), 3.67 (ddd, 1H, J=2.0 3.7 9.1 Hz, H-5), 3.71 (m, 1H, H-5′), 3.94 (dd, 1H, J=5.2 11.7 Hz, H-6b′), 3.99 (dd, 1H, J=9.1 9.3 Hz, H-4), 4.02 (dd, 1H, J=3.6 9.9 Hz, H-3′), 4.20 (m, 1H, H-6b″), 4.23 (m, 1H, 11-3″), 4.39 (m, 2H, H-6ab), 4.54 (d, 1H, J=12.5 Hz, CH₂OPh), 4.57 (d, 1H, J=7.9 Hz, H-1′), 4.64 (d, 1H, J=7.8 Hz, H-1), 4.79 (d, 1H, J=12.5 Hz, CH₂OPh), 5.08 (d, 1H, J=8.1 Hz, H-1″), 5.34 (s, 1H, CHPh), 5.37 (dd, 1H, J=7.8 9.7 Hz, H-2), 5.45 (dd, 1H, J=7.9 9.9 Hz, H-2′), 5.51 (d, 1H, J=3.6 Hz, H-4′), 5.54 (dd, 1H, J=9.1 9.7 Hz, H-3), 6.71 (d, 1H, J=6.4 Hz, NH), 6.85-8.0 (m, 39H, Ph).

¹³C-NMR (CDCl₃, 150 MHz) δ 60.0 (C-2″), 62.0 (C-6′), 62.7 (C-6), 65.9 (C-5″), 68.1 (C-6″), 68.3 (C-3″), 69.2 (C-4′), 70.6 (CH₂OPh), 71.6 (C-5′), 71.7 (C-2), 71.8 (C-2′), 72.9 (C-5), 73.0 (C-3), 75.5 (C-4), 76.7 (C-3′), 81.0 (C-4″), 91.7 (CCl₃), 98.6 (C-1″), 98.8 (C−1), 100.6 (C-1′), 100.9 (CHPh), 127.1-130.0 (Ph), 130.8-131.3 (Ph), 133.6 (Ph), 135.1 (Ph), 135.3 (Ph), 136.3 (Ph), 139.5 (Ph), 139.9 (Ph), 139.9 (Ph), 140.1 (Ph), 140.3 (Ph), 162.2 (COCCl₃), 163.9 (COPh), 164.3 (COPh), 164.5 (COPh), 164.9 (COPh), 165.1 (COPh), 165.2 (COPh).

M.p. 148-150° C. [α]_{D}²²=+27.0 (c=1, CHCl₃).

### Example 8

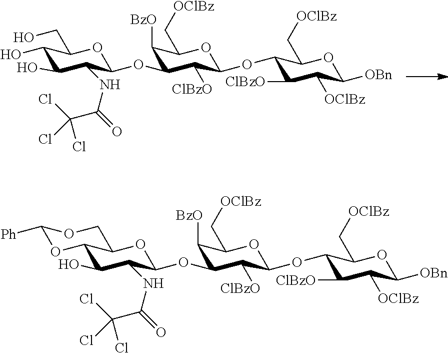

Triol-trisaccharide (5.3 g, 3.45 mmol) was treated with benzaldehyde dimethyl acetal (1.3 mL, 8.63 mmol) and p-TsOH (70 mg, 0.35 mmol) in acetonitrile (30 mL) at 45° C. for 2 hours when the reaction was quenched with Et₃N and evaporated. The benzylidene protected trisaccharide was isolated (4.58 g, 2.81 mmol, 81%) after silica purification (toluene/EtOAc 2.5:1) as a foam.

¹H-NMR (CDCl₃, 300 MHz) δ 2.66 (d, 1H, J=3.3 Hz), 3.03-3.12 (m, 1H), 3.26-3.41 (m, 4H), 3.48 (dd, 1H, J=9.9 9.9 Hz), 3.66-3.74 (m, 3H), 3.92-4.06 (m, 4H), 4.21-4.28 (m, 2H), 4.37-4.38 m, 2H), 4.56 (d, 1H, J=12.3 Hz), 4.58 (d, 1H, J=7.8 Hz), 4.65 (d, 1H, J=7.8 Hz), 4.80 (d, 1H, J=12.3 Hz), 5.11 (d, 1H, J=8.1 Hz), 5.40 (s, 1H), 5.36-5.59 (m, 4H), 6.71 (d, 1H, J=6.6 Hz), 6.85-6.88 (m, 2H), 7.09-7.99 (m, 33H).

¹³C-NMR (CDCl₃, 75.45 MHz) δ 54.4, 59.8, 62.0, 62.7, 65.9, 68.1, 68.2, 69.1, 70.6, 71.5 (2C), 71.6, 72.8, 72.9, 75.4, 80.9, 91.7, 98.6, 98.7, 100.6, 101.7, 126.2, 127.0, 127.4, 127.6, 127.7, 127.8, 128.2, 128.3, 128.4, 128.6, 128.7, 129.0, 129.1, 129.3, 130.0, 130.8, 130.9, 131.1, 131.3, 133.6, 136.2, 136.7, 139.5, 139.8, 140.0, 140.1, 162.1, 163.9, 164.3, 164.5, 164.9, 165.1, 165.2.

### Example 9

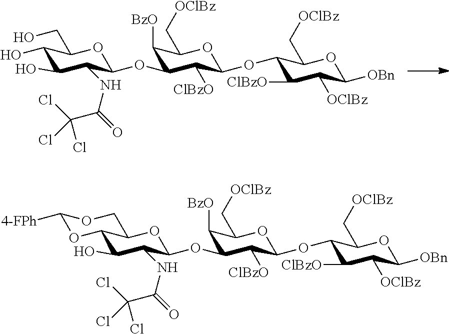

Following the same method as for the benzylidene protected compound above using p-fluorobenzaldehyde dimethyl acetal (2 eq). Yield 75%; amorphous solid.

¹H-NMR (CDCl₃, 300 MHz) δ 2.95-3.035 (m, 1H), 3.18-3.42 (m, 4H), 3.59-3.72 (m, 2H), 3.86-3.99 (m, 3H), 4.13-4.22 (m, 2H), 4.33-4.35 (m, 2H), 4.49 (d, 1H, J=12.5 Hz), 4.51 (d, 1H, J=7.8 Hz), 4.58 (d, 1H, J=7.8 Hz), 4.74 (d, 1H, J=12.5 Hz), 5.02 (d, 1H, J=8.1 Hz), 5.30 (s, 1H), 5.31-5.52 (m, 4H), 6.69 (d, 1H, J=6.6 Hz), 6.78-6.81 (m, 2H), 6.91-7.92 (m, 32H).

### Example 10

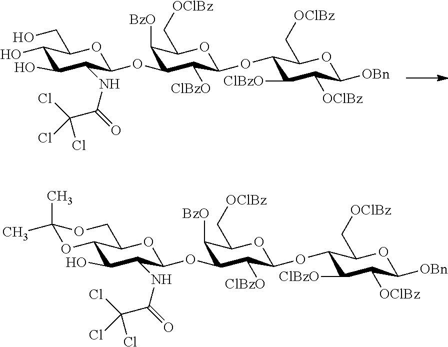

Triol-trisaccharide (1.0 g, 0.65 mmol) was mixed with 2,2′-dimethoxy propane (2 mL), DMF (6 m) and p-TsOH (30 mg) at RT for 2 hours and then quenched with Et₃N followed by evaporation in high vacuum. The isopropylidene protected trisaccharide was isolated as foam (775 mg, 0.49 mmol, 75%) after silica purification (toluene/EtOAc 3:1).

¹H-NMR (CDCl₃, 300 MHz) δ 1.27 (s, 3H), 1.28 (s, 3H), 2.71 (d, 1H, J=3.3 Hz), 2.92-3.12 (m, 2H), 3.22 (dd, 1H, J=9.0 9.0 Hz), 3.29 (dd, 1H, J=7.5 11.7 Hz), 3.38 (dd, 1H, J=10.5 10.5 Hz), 3.58-3.66 (m, 2H), 3.73 (dd, 1H, J=7.5 10.8 Hz), 3.85 (dd, 1H, J=5.4 11.7 Hz), 3.90-3.99 (m, 3H), 4.29-4.38 (m, 2H), 4.48 (d, 1H, J=12.6 Hz), 4.50 (d, 1H, J=7.8 Hz), 4.58 (d, 1H, J=7.5 Hz), 4.73 (d, 1H, J=12.6 Hz), 4.94 (d, 1H, J=8.1 Hz), 5.29-5.40 (m, 2H), 5.42 (d, 1H, J=3.3 Hz), 5.48 (dd, 1H, J=9.6 9.6 Hz), 6.74 (d, 1H, J=6.6 Hz), 6.78-6.81 (m, 2H), 6.90-7.98 (m, 28H).

### Example 11

### Phenyl 2,3,4,6-tetra-O-acetyl-1-thio-β-D-galactopyranoside

To a well stirred suspension of D-galactose (9 g) in acetic anhydride (25 mL) BF₃.OEt₂ (0.62 mL; 0.10 eq). In 2 h the mixture is heated to about 60° C. and to this solution thiophenol (˜8.2 mL) f and BF₃.OEt₂ (1.24 mL; 0.20 eq.) were added. After 6-8 hours the dark orange-colored solution was cooled to RT and poured to a mixture of water (250 mL) and dichloromethane (200 mL). The mixture was stirred for 30 minutes and the phases were separated. The organic phase was washed with saturated NaHCO₃ solution, 0.5 M NaOH solution and saturated NaCl solution, and dried (Na₂SO₄), then the solvent was evaporated. Crystallization from diisopropyl ether gave 13.0 g (59%) white solid. M.p. 79-81° C.

### Example 12

### Methyl 2,3,4,6-tetra-O-acetyl-1-thio-β-D-galactopyranoside

To a mixture of methyl β-D-thio-galactopyranoside (20 g, 95.12 mmol) in pyridine (200 mL) was added acetic anhydride (100 mL) dropwise. The mixture turned into solution and was stirred over night at RT and then concentrated to a syrup. The crude was dissolved in DCM and washed with 1M aq. HCl and then aq. sat. bicarbonate solution. The lower phase was dried over sodium sulphate, filtered and concentrated. The obtained syrup was dissolved in hot ethanol (250 mL) and left with stirring to get the target compound as white crystals (31.3 g, 87%). NMR confirm to structure.

### Example 13

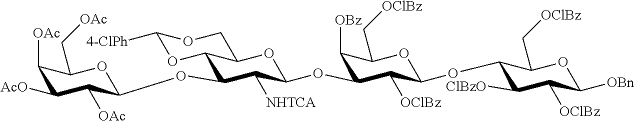

To solution of phenyl 2,3,4,6-tetra-O-acetyl-1-thio-β-D-galactopyranoside (3.44 g) and the appropriate acceptor (10.0 g) in DCM (40 mL)N-bromosuccinimide (1.53 g) was added at RT. After 10 minutes TfOH (26 μL) was added drop wise. After 15 min. at RT the reaction was quenched with the addition of aq. NH₄OH (1.5 mL, 25%). The DCM phase was washed twice with aq. Na₂S₂O₃ (10%)/sat. aq. NaHCO₃ mixture (2:1, 2×80 mL) and then brine/water mixture (1:1, 2×80 mL). The crude product in DCM was taken directly into the next step without further purification.

¹H-NMR (CDCl₃, 300 MHz) δ 1.75 (s, 3H), 1.91 (s, 6H), 2.06 (s, 3H), 3.01-3.10 (m, 1H), 3.37-3.52 (m, 4H), 3.56 (dd, 1H, J=5.4 5.4 Hz), 3.65-3.74 (m, 2H), 3.83 (dd, 1H, J=6.3 11.1 Hz), 3.90-4.06 (m, 4H), 4.21 (dd, 1H, J=4.2 9.9 Hz), 4.35-4.45 (m, 2H), 4.48-4.58 (m, 4H), 4.66 (d, 1H, J=7.8 Hz), 4.79-4.83 (m, 2H), 5.04 (dd, 1H, J=7.8 10.2 Hz), 5.19 (d, 1H, J=8.1 Hz), 5.23 (d, 1H, J=3.3 Hz), 5.38 (s, 111), 5.37-5.47 (m, 2H9, 5.52-5.60 (m, 2H), 6.78 (d, 1H, J=6.9 Hz), 6.89 (m, 2H), 7.09-8.00 (m, 32).

¹³C-NMR (CDCl₃, 75.45 MHz) δ 20.4, 20.5 (2C), 20.6, 59.4, 61.0, 61.9, 62.7, 65.9, 66.7, 68.1, 68.8, 69.1, 70.5, 70.5, 70.8, 71.4, 71.4, 71.6, 72.8, 72.9, 74.7, 75.4, 76.6, 78.2, 91.6, 98.1, 98.7, 98.9, 100.5, 100.7, 127.1-129.9, 129.9, 130.7-131.3, 133.6, 135.1, 135.3, 136.2, 139.5, 139.8, 140.0, 140.0, 140.2, 161.8, 163.6, 164.2, 164.4, 164.8, 165.0, 165.1, 169.1, 169.9, 170.0, 170.1.

[α]_{D}²²=+21.7 (c=1, CHCl₃).

### Example 14

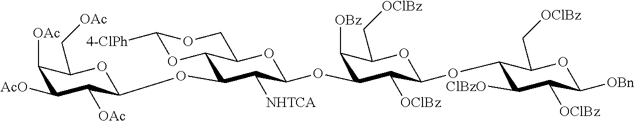

Methyl 2,3,4,6-tetra-O-acetyl-1-thio-β-D-galactopyranoside (93 mg, 0.246 mmol) and benzylidene trisaccharide acceptor (200 mg, 0.123 mmol) were dissolved in dry DCM (4 mL) and cooled to −15° C. under argon atmosphere. N-Iodosuccinimide (55 mg) and AgOTf (15 mg) were added in one portion. After 15 minutes the reaction was quenched with Et₃N, diluted with DCM and sequent washed with aq. sodium thiosulfate solution and brine, before dried over sodium sulphate, filtered and concentrated. The product was isolated as an amorphous solid (165 mg, 69%) by silica purification using toluene/EtOAc (2:1) as eluent.

¹H-NMR (CDCl₃, 300 MHz) δ 1.71 (s, 3H), 1.89 (s, 3H), 1.90 (s, 3H), 2.05 (s, 3H), 3.02-3.10 (m, 1H), 3.36-3.58 (m, 5H), 3.65-3.74 (m, 2H), 3.82 (dd, 1H, J=6.6 11.1 Hz), 3.89-4.07 (m, 4H), 4.22 (dd, 1H, J=4.2 10.2 Hz), 4.35-4.62 (m, 6H), 4.66 (d, 1H, J=7.8 Hz), 4.79-4.83 (m, 2H), 5.03 (dd, 1H, J=8.1 10.5 Hz), 5.21-5.23 (m, 2H), 5.41 (s, 1H), 5.37-5.47 (m, 2H), 5.53 (d, 1H, J=3.3 Hz), 5.57 (dd, 1H, J=9.6 9.6 Hz), 6.82 (d, 1H, J=6.6 Hz), 6.87-6.90 (m, 2H), 7.09-7.99 (m, 32H).

¹³C-NMR (CDCl₃, 75.45 MHz) δ 20.4, 20.5 (3C), 59.3, 61.1, 61.9, 62.6, 65.9, 66.7, 68.1, 68.8, 69.1, 70.4, 70.5, 70.8, 71.4 (2C), 71.6, 72.8, 72.9, 74.7, 75.4, 76.7, 78.1, 91.6, 98.1, 98.6, 98.7, 100.7, 101.3, 126.0, 127.1, 127.4, 127.5, 127.7, 127.8, 128.0, 128.1, 128.2, 128.3, 128.4, 128.6, 128.7, 129.0, 129.3, 129.9, 130.7, 130.8, 130.9, 131.1, 131.3, 133.6, 136.2, 136.7, 139.5, 139.8, 140.0 (2C), 140.1, 161.8, 163.7, 164.2, 164.4, 164.8, 165.0, 165.1, 169.2, 169.9, 170.0, 170.1.

### Example 15

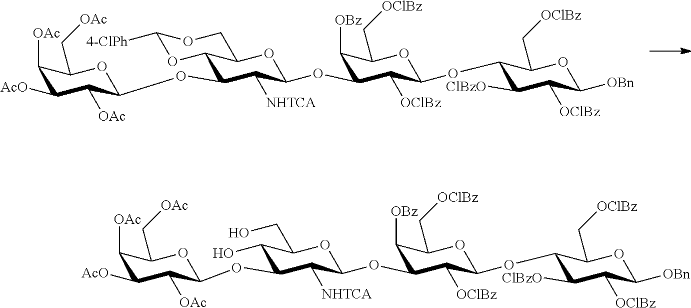

The crude benzylidene tetrasaccharide in DCM (40 mL) was cooled to 0-5° C. using. Diluted aq. HClO₄ (2 mL, 1 part 70% aq. HClO₄ diluted with 1 part water) was added drop wise and the solution was stirred vigorously at 0-5° C. for 2 hours when aq. sat. NaHCO₃ (20 mL) was added and stirred for half an hour. Water (20 mL) was added and the phases were separated. The lower organic phase was washed with brine/water mixture (40 mL) and concentrated. The residue was crystallized by adding TBME (40 mL) to give 7.7 g (73%) of the diol.

¹H-NMR (CDCl₃, 600 MHz) δ 1.89 (s, 3H, CH₃COO), 1.93 (s, 3H, CH₃COO), 2.00 (s, 3H, CH₃COO), 2.08 (s, 3H, CH₃COO), 2.92 (ddd, 1H, 3.29 (m, 1H, H-5″), 3.30 (m, 1H, H-4″), 3.51 (m, 1H, H-6a″), 3.53 (m, 1H, H-6a′), 3.60 (ddd, 1H, J=1.9 4.7 9.8 Hz), 3.68 (m, 1H, H-5′), 3.68 (m, 1H, H-6b′), 3.76 (m, 1H, H-6b″), 3.87 (m, 1H, H-5″), 3.95 (dd, 1H, J=3.5 9.9 Hz, H-3′), 4.00 (m, 1H, H-6a′″), 4.01 (dd, 1H, J=9.6 9.8 Hz, H-4), 4.05 (m, H-6b′″), 4.23 (dd, 1H, J=8.7 10.1 Hz, H-3″), 4.32 (m, 1H, H6a), 4.37 (d, 1H, J=8.1 Hz, H-1′″), 4.39 (m, 1H, H-6b), 4.53 (d, 1H, J=11.9 Hz, CH₂OPh), 4.57 (d, 1H, J=7.8 Hz, H-1′), 4.63 (d, 1H, J=8.1 Hz, H-1), 4.76 (dd, 1H, J=3.5 10.5 Hz, H-3″), 4.78 (d, 1H, J=11.9 Hz, CH₂OPh), 5.08 (dd, 1H, J=8.1 10.5 Hz, H-2″), 5.12 (d, 1H, J=8.1 Hz, H-1″), 5.27 (dd, 1H, J=0.9 3.5 Hz, H-4″), 5.37 (dd, 1H, J=8.1 9.6 Hz, H-2), 5.43 (dd, 1H, J=7.8 9.9 Hz, H-2′), 5.53 (dd, 1H, J=9.1 9.6 Hz, H-3), 5.75 (d, 1H, J=3.5 Hz, H-4′), 6.84 (d, 1H, J=8.5 Hz, NH), 7.09-8.05 (m, 30H, Ph).

¹³C-NMR (CDCl₃, 150 MHz) δ 20.4 (CH₃COO), 20.5 (CH₃COO), 20.5 (CH₃COO), 20.9 (CH₃COO), 58.9 (C-2″), 61.3 (C-6″), 61.4 (C-6′), 61.6 (C-6″), 62.6 (C-6), 66.7 (C-4′″), 68.4 (C-4″), 68.6 (C-2″), 69.8 (C-4′), 70.6 (CH₂Ph), 70.7 (C-3′″), 71.1 (C-5′), 71.2 (C-5′″), 71.4 (C-2′), 71.6 (C-2), 72.8 (C-5), 73.0 (C-3), 75.2 (C-4), 76.1 (C-5″), 78.3 (C-3′), 81.0 (C-3″), 91.3 (NHCOCCl₃), 98.8 (C−1), 98.8 (C-1″), 100.5 (C-1″), 100.6 (C-1′), 127.8 (Ph), 128.1 (Ph), 128.4 (Ph), 128.8-129.1 (Ph), 130.2 (Ph), 130.8 (Ph), 130.9 (Ph), 131.0 (Ph), 131.2 (Ph), 131.6 (Ph), 134.0 (Ph), 161.8 (NHCOCCl₃), 164.0 (COOPh), 164.2 (COOPh), 164.5 (COOPh), 164.8 (COOPh), 165.1 (COOPh), 166.2 (COOPh), 169.2 (CH₃COO), 169.9 (CH₃COO), 170.0 (CH₃COO), 170.4 (CH₃COO).

M.p. 150-152° C. [α]_{D}²²=+23.8 (c=1, CHCl₃).

### Example 16

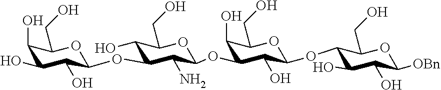

LNT diol (20 g, 10.69 mmol) was suspended in 100 mL of MeOH by heating. Methanolic NaOMe solution (800 μL, 25% by wt in MeOH) was added. After approximately 20 min a precipitate starts to form and after 1 hour a thick suspension is obtained in the flask. After 5 hours the reaction is quenched by the addition of 150 μL of glacial AcOH. The methanol was evaporated off on the rotational evaporator and then 40 mL of water and 40 mL of mixed hexanes were added. The phases were separated and the lower phase holding the product was washed again with mixed hexanes (40 mL). The lower phase was taken to the rotational evaporator and vacuum was applied to remove residual MeOH and hexanes. The obtained aq. extract holding the deprotected NHTCA-OBn tetra-saccharide was used directly in the next NHTCA hydrolysis step without further purification.

The NHTCA-OBn tetra-saccharide in water was heated to 55° C. and 640 μL of NaOH solution (1 g/mL) was added. After 4 hours the reaction solution was cooled to 15° C. and MeOH (50 mL) was added. The precipitation of the product started immediately. Another 25 mL of MeOH was added followed by isopropanol (15 mL). The precipitation was filtered off and dried to give 6.84 g (85%) of the LNT amine as a white solid.

¹H-NMR (D₂O, 400 MHz) δ 2.89 (dd, 1H, 8.4 8.6 Hz, H-2), 3.36 (dd, 1H, J=7.9 8.7 Hz, H-2), 3.47 (ddd, 1H, J=2.1 4.9 9.6 Hz, H-5″), 3.54 (dd, 1H, J=8.4 9.6 Hz, H-4″), 3.59 (m, 1H, H-5), 3.59 (dd, 1H, J=8.4 8.6 Hz, H-3″), 3.61 (m, 1H, 11-2″), 3.62 (dd, 1H, J=8.7 8.9 Hz, H-3), 3.67 (dd, 1H, J=8.3 8.9 Hz, H-4), 3.68 (dd, 1H, J=7.9 9.8 Hz, H-2′), 3.69 (m, 1H, H-3″), 3.73 (m, 1H, H-5′), 3.75 (m, 1H, H-5″), 3.76 (m, 2H, H-6ab′″), 3.76 (m, 2H, H-6ab′), 3.77 (dd, 1H, J=4.9 13.0 Hz, H-6a″), 3.81 (dd, 1H, J=3.3 9.8 Hz, H-3′), 3.82 (m, 1H, H-6a), 3.88 (dd, 1H, J=2.1 13.0 Hz, H-6b″), 3.92 (d, 1H, J=3.3 Hz, H-4″), 4.00 (dd, 1H, J=2.0 12.5 Hz, H-6b), 4.18 (d, 1H, J=3.3 Hz, H-4′), 4.49 (d, 111, J=7.9 Hz, H-1′), 4.54 (d, 1H, J=7.6 Hz, H-1″), 4.56 (d, 1H, J=7.9 Hz, H-1), 4.63 (d, 1H, J=8.4 Hz, H-1″), 4.76 (d, 1H, J=11.7 Hz, CH₂Ph), 4.94 (d, 1H, J=11.7 Hz, CH₂Ph), 7.40-7.50 (m, 5H, Ph).

¹³C-NMR (D₂O, 100 MHz) δ 58.9 (C-2″), 62.8 (C-6), 63.2 (C-6″), 63.6 (C-6′), 63.6 (C-6″), 70.8 (C-4″), 70.9 (C-4′), 71.2 (C-4″), 72.8 (C-2′), 73.8 (C-2″), 74.2 (CH₂Ph), 75.3 (C-3″), 75.5 (C-2), 77.1 (C-3), 77.4 (C-5), 77.7 (C-5′), 78.1 (C-5″), 78.2 (C-5″), 81.1 (C-4), 84.9 (C-3′), 89.4 (C-3″), 103.7 (C−1), 105.4 (C-1′), 106.7 (C-1″), 106.9 (C-1″), 131.2 (Ph), 131.4 (2C, Ph)), 131.4 (2C, Ph), 139.2 (Ph).

M.p. 245° C. (dec.). [α]_{D}²²=−2.8 (c=1, H₂O).

### Example 17

### 1-O-benzyl-β-LNT

To LNT amine (50 g) water was added (150 mL) and to this suspension was added acetic anhydride (8.11 mL) drop wise at RT. A clear solution was obtained which was stirred for 30 minutes at RT whereupon aq. 50% NaOH solution (4.6 mL) was added to neutralize the pH. Another portion of acetic anhydride (1.26 mL) was added drop wise and the mixture was stirred further 30 minutes. Aq. NaOH (3.86 mL, 50%) was added to get a pH above 11 and the solution was heated to 45° C. for 30 minutes. MeOH (600 mL) was added in one portion and the solution was heated to 50-55° C. At this temperature acetone (425 mL) was added and the product started to precipitate. After complete addition of the acetone the suspension was cooled to RT and stirred for 30 minutes. The compound was filtered off, washed with methanol and dried to get 1-O-benzyl-β-LNT (45.39 g, 86%) as a white solid.

¹H-NMR (D₂O, 400 MHz) 6 2.03 (s, 3H, CH₃CONH), 3.35 (dd, 1H, J=8.1 8.5 Hz, H-2), 3.49 (m, 1H, H-5″), 3.53 (m, H-2″), 3.65 (m, 1H, H-3″), 3.57 (dd, 1H, J=8.1 9.0 Hz, H-4″), 3.58 (m, 1H, H-5), 3.59 (dd, 1H, J=7.7 10.0 Hz, H-2′), 3.62 (m, 1H, H-3), 3.63 (m, 1H, H-4), 3.71 (m, 1H, H-5′), 3.71 (m, 1H, H-5′″), 3.73 (dd, 1H, J=3.3 10.0 Hz, H-3′), 3.76 (m, 2H, H-6ab′″), 3.76 (m, 2H, H-6ab′), 3.80 (m, 1H, H-6a″), 3.80 (dd, 1H, J=5.0 12.2 Hz, H-6a), 3.82 (dd, 1H, J=8.1 10.5 Hz, H-3″), 3.90 (m, 1H, H-6b″), 3.90 (dd, 1H, J=8.4 10.5 Hz, H-2″), 3.92 (d, 1H, J=3.3 Hz, H-4″), 3.98 (dd, 1H, J=1.6 12.2 Hz, H-6b), 4.15 (d, 1H, J=3.3 Hz, H-4′), 4.44 (d, 1H, J=7.7 Hz, H-1′), 4.45 (d, 1H, J=7.7 Hz, H-1′″), 4.56 (d, 1H, J=8.1 Hz, H-1), 4.73 (d, 1H, J=8.4 Hz, H-1″), 4.76 (d, 1H, J=11.7 Hz, CH₂Ph), 4.94 (d, 1H, J=11.7 Hz, CH₂Ph), 7.40-7.50 (m, 5H, Ph).

¹³C-NMR (D₂O, 100 MHz) 6 24.9 (CH₃CONH), 57.4 (C-2″), 62.8 (C-6), 63.2 (C-6″), 63.7 (C-6′″), 63.7 (C-6′), 71.0 (C-4′), 71.2 (C-4′″), 71.3 (C-4″), 72.7 (C-2′), 73.4 (C-2″), 74.2 (CH₂Ph), 75.2 (C-3′″), 75.5 (C-2), 77.1 (C-3), 77.5 (C-5′), 77.6 (C-5′″), 77.9 (C-5), 78.0 (C-5″), 81.1 (C-4), 84.7 (C-3′), 84.8 (C-3″), 103.7 (C−1), 105.3 (C-1″), 105.6 (C-1′), 106.2 (C-1′″), 131.1 (Ph), 131.4 (2C, Ph), 131.5 (2C, Ph), 139.2 (Ph), 177.7 (CH₃CONH).

M.p. 245° C. (dec.). [α]_{D}²²=−10.3 (c=1, H₂O).

### Example 18

### LNT

1-O-benzyl-β-LNT (5 g, 6.27 mmol) was suspended in water (20 mL) and the pH was adjusted to 5.8 by addition of 1M aq. HCl. Palladium on charcoal (0.5 g, 10%) was added and the reaction flask was evacuated and then saturated with H₂ (4 bar). The reaction temperature was set to 50° C. and after stirring for 1.5 hour the temperature was allowed to reach RT, the catalyst was removed by filtration and water was used for washing (10 mL). The filtrate was concentrated to dryness and 3.46 g (78%) of white solid was obtained.

¹H-NMR (D₂O, 300 MHz) δ 1.88 (s, 3H), 3.13 (m, 1H), 3.31-3.82 (m, 22H), 4.00 (d, 1H, J=3.3 Hz), 4.30 (d, 2H, J=7.8 Hz), 4.51 (d, 1H, J=8.1 Hz), 4.58 (d, 1H, J=8.1 Hz, H-1β), 5.07 (d, 1H, J=3.6 Hz, H-1α). ¹³C-NMR (D₂O, 75.45 MHz, some signals are overlapping) 8 22.4, 54.9, 60.2, 60.6, 61.1, 61.2, 68.5, 68.6, 68.7, 70.2, 70.3, 70.8, 71.3, 71.6, 72.6, 73.9, 74.5, 74.9, 75.1, 75.3, 75.4, 78.4, 78.5, 82.1, 82.0, 92.0 (C-1α), 95.9 (C-1β), 102.7, 103.1, 103.6, 175.1.

Having thus described the present invention in detail and the advantages thereof, it is to be understood that the detailed description is not intended to limit the scope of the invention thereof.

What is desired to be protected by letters patent is set forth in the following claims.

1. A method for the manufacture of Galpβ1-3GlcNAcpβ1-3Galpβ1-4Glc (LNT), comprising a catalytic hydrogenolysis of a compound of general formula 1 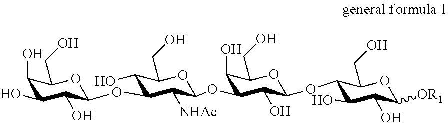
wherein R₁ is a group removable by catalytic hydrogenolysis.

2. The method according to claim 1, wherein the catalytic hydrogenolysis is carried out in water or in aqueous alcohol under a hydrogen atmosphere in the presence of Pd on charcoal or Pd black.

3. The method according to claim 1, wherein R₁ in the compound of general formula 1 is benzyl.

4. The method according to claim 1, wherein the compound of general formula 1 is crystalline.

5. A method according to claim 1, wherein the compound of general formula 1 is obtained by a conversion of a compound of general formula 6 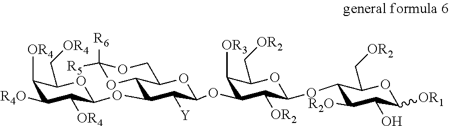
wherein R₁ is as defined in claim 1,
R₂ is optionally substituted acyl,
R₃ is H or optionally substituted acyl,
R₄ is optionally substituted acyl,
R₅ is alkyl or optionally substituted phenyl,
R₆ is H, alkyl or optionally substituted phenyl, or
R₅ and R₆ with the carbon atom to which they are attached form a C₃-C₆ cycloalkyl ring, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
by one or more deprotection steps.

6. The method according to claim 5, wherein the compound of general formula 1 is isolated as crystalline.

7. The method according to claim 5, comprising
a) an acid catalyzed hydrolysis of the compound of general formula 6 defined in claim 5 to a compound of general formula 5 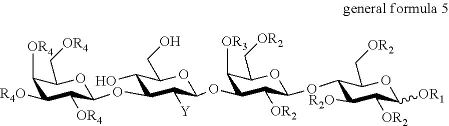
wherein R₁, R₂, R₃ R₄ and Y are as defined in claim 5, and
b) a further conversion of the compound of general formula 5 obtained above by one or more deprotection steps into a compound of general formula 1 defined in claim 1.

8. The method according to claim 7, comprising a base catalyzed transesterification reaction or a basic hydrolysis of a compound of general formula 5 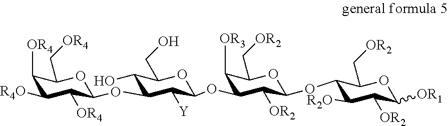
wherein R₁, R₂, R₃ and R₄ are as defined in claim 7, and
Y is —NHAc or —NAc₂,
to obtain the compound of general formula 1 defined in claim 1.

9. The method according to claim 7, comprising:
a) a conversion of a compound of general formula 5 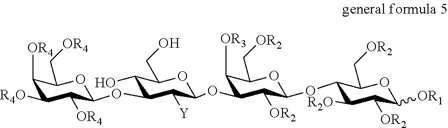
wherein R₁, R₂, R₃ and R₄ are as defined in claim 7, and
Y is selected from alkanoylamido, haloalkanoylamido, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
by one or more deprotection steps into a compound of general formula 3 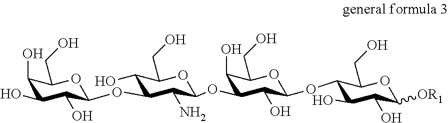
wherein R₁ is as defined in claim 7, and
b) a conversion of the compound of general formula 3 into the compound of general formula 1 defined in claim 1 by:
ba) a selective N-acetylation of the compound of general formula 3, or
bb) a peracetylation of the compound of general formula 3 to a compound of general formula 2 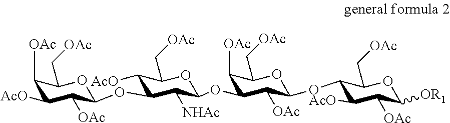
wherein R₁ is as defined above,
followed by a base catalyzed transesterification reaction or a basic hydrolysis.

10. The method according to claim 9, wherein the conversion of the compound of general formula 5 into the compound of general formula 3 in step a) comprises:
a) a base catalyzed transesterification deprotection of R₂, R₃, and R₄ of the compound of general formula 5 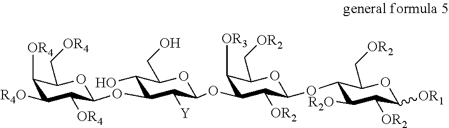
wherein R₁, R₂, R₃ and R₄ are as defined in claim 7, and
Y is selected from alkanoylamido (with the proviso that acetamido is excluded), haloalkanoylamido, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
to form a compound of general formula 4 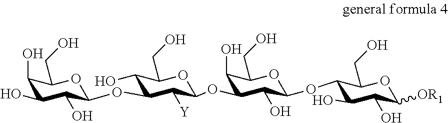
wherein R₁ and Y are as defined above,
which compound of general formula 4 is then subjected to
aa) a basic hydrolysis when Y is selected from haloalkanoylamido, 2,3-diphenylmaleimido and 2,3-dimethylmaleimido, or
ab) an aminolysis when Y is selected from alkanoylamido (with the proviso that acetamido is excluded), haloalkanoylamido, alkoxycarbonylamino, haloalkoxycarbonylamino, benzyloxycarbonylamino, optionally substituted benzamido, phthalimido, tetrachlorophthalimido, 2,3-diphenylmaleimido and 2,3-dimethylmaleimido, or
ac) a Zn/HCl treatment when Y is 2,2,2-trichloroetoxycarbonylamino, or
ad) a catalytic hydrogenolysis when Y is benzyloxycarbonylamino or azido to give a compound of general formula 3; or
b) basic hydrolysis of the compound of general formula 5, wherein R₁, R₂, R₃ and R₄ are as defined in claim 7, and Y is selected from haloalkanoylamido, 2,3-diphenylmaleimido and 2,3-dimethylmaleimido, or
c) an aminolysis of the compound of general formula 5, wherein R₁, R₂, R₃ and R₄ are as defined in claim 7, and Y is selected from alkanoylamido, haloalkanoylamido, alkoxycarbonylamino, haloalkoxycarbonylamino, benzyloxycarbonylamino, optionally substituted benzamido, —NAc₂, phthalimido, tetrachlorophthalimido, 2,3-diphenylmaleimido and 2,3-dimethylmaleimido.

11. The method according to claim 5, further comprising a reaction of a donor of general formula 8 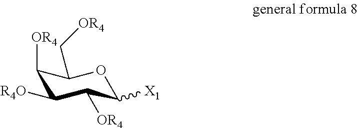
wherein R₄ is optionally substituted acyl, and
X₁ is selected from halogen, —OC(═NH)CCl₃, —OAc, —OBz and —SR₇, wherein R₇ is selected from alkyl and optionally substituted phenyl,
with an acceptor of general formula 7 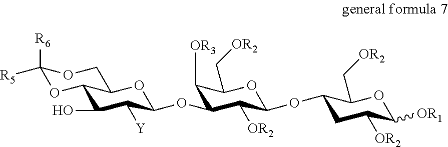
wherein R₁ is as defined in claim 1,
R₂ is optionally substituted acyl,
R₃ is H or optionally substituted acyl,
R₅ is alkyl or optionally substituted phenyl,
R₆ is H, alkyl or optionally substituted phenyl, or
R₅ and R₆ with the carbon atom to which they are attached form a C₃-C₆ cycloalkyl ring, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
to give the compound of general formula 6 as defined in claim 5.

12. The method according to claim 11, further comprising a reaction of a compound of general formula 9 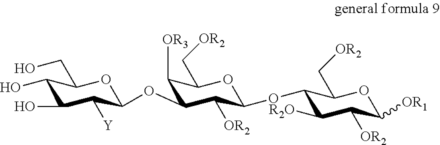
wherein R₁, R₂, R₃ and Y are as defined in claim 11,
with an aldehyde or ketone of formula R₅R₆C═O or di-O-alkyl-acetal/ketal thereof,
wherein R₅ is selected from alkyl or optionally substituted phenyl,
R₆ is selected from H, alkyl or optionally substituted phenyl, and
R₅ and R₆ with the carbon atom to which they are attached form a C₃-C₆ cycloalkyl ring,
under acid catalysis to form a compound of general formula 7 defined in claim 11.

13. The method according to claim 12, further comprising a selective acidic deacetylation of a compound of general formula 10 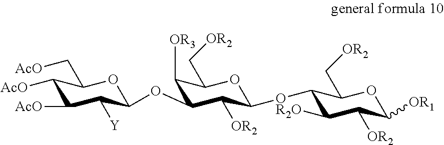
wherein R₁, R₂, R₃ and Y are as defined in claim 12,
to yield the compound of general formula 9 as defined in claim 12.

14. The method according to claim 13, further comprising the reaction of a donor of general formula 12 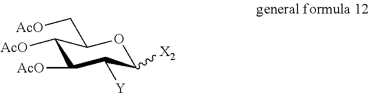
wherein X₂ is selected from halogen, —OC(═NH)CCl₃, —OAc, —OBz or —SR₇, in which R₇ is alkyl or optionally substituted phenyl,
Y is as defined in claim 13, or
Y with the vicinal X₂ forms 2-alkyl-, 2-haloalkyl- or 2-(optionally substituted phenyl)-oxazoline,
with an acceptor of general formula 11 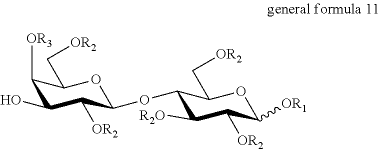
wherein R₁, R₂ and R₃ are as defined in claim 13,
to give the compound of general formula 10 defined in claim 13.

15. A compound of general formula 1 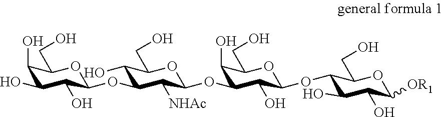
wherein R₁ is a group removable by catalytic hydrogenolysis,
characterized by a crystalline form.

16-24. (canceled)

25. A compound of general formula A 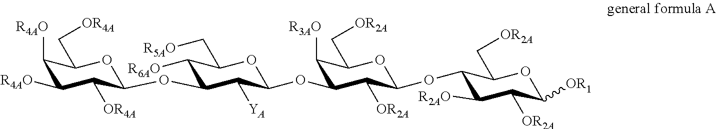
wherein R₁ is a group removable by catalytic hydrogenolysis,
R_{2A} is selected from the group of optionally substituted acyl and H,
R_{3A} is selected from the group of optionally substituted acyl and H,
R_{4A} is selected from the group of optionally substituted acyl and H,
R_{5A} is H,
R_{6A} is H, or
R_{5A} and R_{6A} together form a moiety 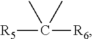
wherein R₅ is alkyl or optionally substituted phenyl, R₆ is H, alkyl or optionally substituted phenyl, or R₅ and R₆ with the carbon atom to which they are attached form a C₃-C₆ cycloalkyl ring, and
Y_{A} is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido, 2,3-dimethylmaleimido and —NH₂,
provided that if Y_{A} is —NHAc then R_{2A}, R_{3A}, R_{4A}, R_{5A} and R_{6A} cannot be H in the same time, preferably the compound of general Formula A is characterized by general formula 2 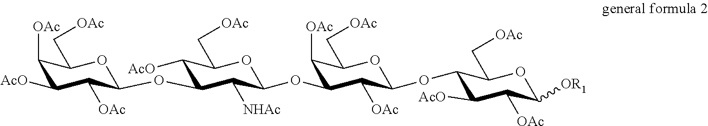
wherein R₁ is a group removable by catalytic hydrogenolysis, or the compound of general Formula A is characterized by general formula 3 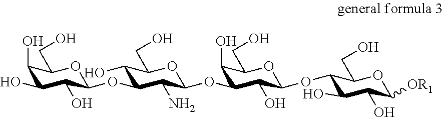
wherein R₁ is a group removable by catalytic hydrogenolysis, or the compound of general Formula A is characterized by general formula 4 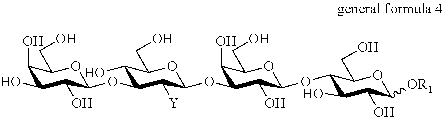
wherein R₁ is a group removable by catalytic hydrogenolysis, and
Y is selected from alkanoylamido, haloalkanoylamido, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido, or the compound of general Formula A is characterized by general formula 5 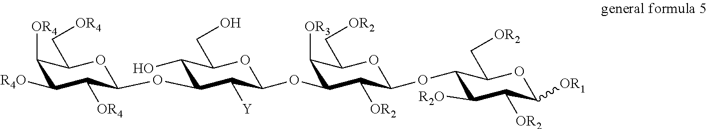
wherein R₁ is a group removable by catalytic hydrogenolysis,
R₂ is optionally substituted acyl,
R₃ is H or optionally substituted acyl,
R₄ is optionally substituted acyl, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido, or the compound of general Formula A is characterized by general formula 6 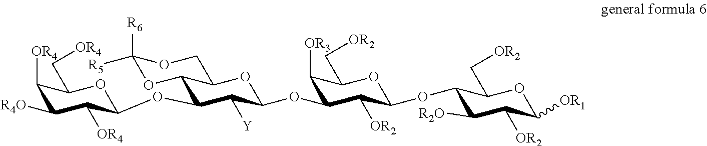
wherein R₁ is a group removable by catalytic hydrogenolysis,
R₂ is optionally substituted acyl,
R₃ is H or optionally substituted acyl,
R₄ is optionally substituted acyl,
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
R₅ is alkyl or optionally substituted phenyl,
R₆ is H, alkyl or optionally substituted phenyl, or
R₅ and R₆ with the carbon atom to which they are attached form a C₃-C₆ cycloalkyl ring.

26. A compound of general formula 7 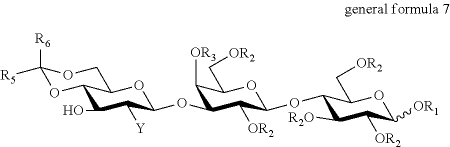
wherein R₁ is a group removable by catalytic hydrogenolysis,
R₂ is optionally substituted acyl,
R₃ is H or optionally substituted acyl,
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido,
R₅ is alkyl or optionally substituted phenyl,
R₆ is H, alkyl or optionally substituted phenyl, or
R₅ and R₆ with the carbon atom to which they are attached form a C₃-C₆ cycloalkyl ring.

27. A compound of general formula 9 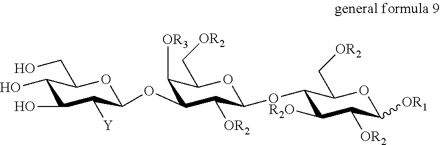
wherein R₁ is a group removable by catalytic hydrogenolysis,
R₂ is optionally substituted acyl,
R₃ is H or optionally substituted acyl, and
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.

28. A compound of general formula 10 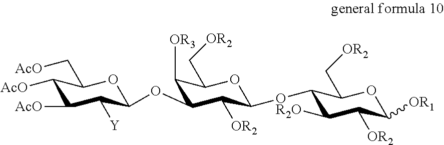
wherein R₁ is a group removable by catalytic hydrogenolysis,
R₂ is optionally substituted acyl,
R₃ is H or optionally substituted acyl,
Y is selected from alkanoylamido, haloalkanoylamido, —NAc₂, alkoxycarbonylamino, haloalkoxycarbonylamino, benzyloxycarbonylamino, azido, optionally substituted benzamido, phthalimido, tetrachlorophthalimido, 2,3-diphenylmaleimido and 2,3-dimethylmaleimido.
